SEQUENCE LISTING (10 sequences)

SEQ ID NO: 1 - DNA, 18 bp, Artificial Sequence
  primer
cgtccgcctctggcagta

SEQ ID NO: 2 - DNA, 21 bp, Artificial Sequence
  primer
tggaagagtttgcagagcagg

SEQ ID NO: 3 - DNA, 19 bp, Artificial Sequence
  primer
cggccctggaacttcggcg

SEQ ID NO: 4 - DNA, 21 bp, Artificial Sequence
  primer
agcccagcagagaaaggaatc

SEQ ID NO: 5 - DNA, 19 bp, Artificial Sequence
  primer
agccagccttccagagctc

SEQ ID NO: 6 - DNA, 27 bp, Artificial Sequence
  primer
aagaagccaccagctaaactgcagcca

SEQ ID NO: 7 - protein, 16 aa, Homo sapien
QQRKESKKPPAKLQPR

SEQ ID NO: 8 - DNA, 10 bp, Unknown
  NF-kappaB-like site
ggaatttcct

SEQ ID NO: 9 - DNA, 22 bp, Artificial Sequence
  primer
gcctctcacctccctctctttc

SEQ ID NO: 10 - DNA, 21 bp, Artificial Sequence
  primer
ctcgcaatgtgctaggtcatg

(19) United States
(12) Patent Application Publication
(10) Pub. No.: US 2007/0025991 A1
(43) Pub. Date: Feb. 1, 2007

(54) Use of antagonists of ghrelin or ghrelin receptor to treat intestinal inflammation

(76) Inventors: Charalabos Pothoulakis, Waban, MA (US); Christos S. Mantzoros, Watertown, MA (US); Dezheng Zhao, Newton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/549,688

(22) PCT Filed: Mar. 19, 2004
(86) PCT No.: PCT/US04/08385

§ 371 Date: Jul. 27, 2006

Related U.S. Application Data
(60) Provisional application No. 60/456,090, filed on Mar. 19, 2003.

(51) Int. Cl. A61K 39/395; A61K 38/17; A61K 38/22

(52) U.S. Cl. 424/145.1; 514/12

(57) ABSTRACT

Methods for treating intestinal inflammation or ghrelin-mediated inflammation by inhibiting the activity of ghrelin or its receptor are described. Also described are methods for identifying ghrelin antagonists and ghrelin receptor antagonists.

### RELATED APPLICATION

This application claims the benefit of U.S. Provisional Application No. 60/456,090, filed on Mar. 19, 2003. The entire teachings of the above application are incorporated herein by reference.

### BACKGROUND OF THE INVENTION

Intestinal (gut) mucosa is normally infiltrated with a large number of mononuclear cells (Monteleone, I. et al., Gut, 50(Suppl III):iii60-iii64 (2002)). This 110 characteristic state of physiological inflammation of the gastrointestinal tract is a tightly controlled phenomenon, as several mucosal cells interact to generate and maintain an appropriate local immune response (Monteleone, I. et al., Gut, 50(Suppl III):iii60-iii64 (2002)). Changes in cell type number and/or function, including release of soluble mediators, have been associated with the development of chronic (pathological) intestinal inflammation (Monteleone, I. et al., Gut, 50(Suppl III):iii60-iii64 (2002)). Chronic intestinal inflammation has been associated with the development of chronic inflammatory diseases such as inflammatory bowel disease (IBD) (e.g., Crohn's disease and ulcerative colitis). Such inflammation may also be associated with parasitic infections, autoimmune inflammation or response, acute enterocolitis or chronic enterocolitis. The inflammation may be mediated by a bacterium, a virus, a parasite or a toxin (e.g., a toxin produced by Clostridium difficile).

### SUMMARY OF THE INVENTION

The present invention provides methods of inhibiting or decreasing an inflammatory response in intestinal tissue comprising administering an effective amount of an agent such as, for example, a ghrelin or ghrelin receptor antagonist, thereby inhibiting the inflammatory response. Administering the agent can be by means of directly contacting intestinal tissue cells with the agent or by delivering the agent alone or in a composition with an acceptable carrier or delivery vehicle. The methods of the present invention encompass methods of inhibiting or decreasing intestinal inflammation in a mammal (e.g., a human) by administering an effective amount of an agent such as, for example, a ghrelin or ghrelin receptor antagonist, to inhibit or decrease intestinal inflammation.

The invention also provides methods of inhibiting or decreasing a ghrelin-mediated inflammatory response comprising administering an effective amount of an agent such as, for example, a ghrelin or ghrelin receptor antagonist, thereby inhibiting the ghrelin-mediated inflammatory response. The methods of the invention encompass methods of inhibiting or decreasing ghrelin-mediated inflammation in a mammal by administering an effective amount of an agent such as, for example, a ghrelin or ghrelin receptor antagonist, to inhibit or decrease ghrelin-mediated inflammation.

The present invention encompasses methods of treating intestinal inflammation (gut inflammation) comprising inhibiting or modulating ghrelin activity, ghrelin binding to the ghrelin receptor or the signaling activity of the ghrelin receptor. The present invention also encompasses methods for treating ghrelin-mediated inflammation (i.e., inflammation associated with upregulation of ghrelin or ghrelin receptor) comprising inhibiting or modulating ghrelin activity, ghrelin binding to the ghrelin receptor or the signaling activity of the ghrelin receptor. The methods disclosed herein contemplate the use of an agent that inhibits, i.e., inhibitors or antagonists, or modulates, e.g., agonists or other effectors, the activity of ghrelin or the ghrelin receptor such that inflammation is reduced or inhibited. In particular, the methods disclosed herein comprise the use of ghrelin antagonists, e.g., ghrelin antibodies, ghrelin derivatives, and small molecules; ghrelin inhibitors, e.g., small molecules, ghrelin receptor peptides or fragments, and ghrelin antibodies; ghrelin receptor inhibitors, e.g., small molecules, ghrelin receptor antibodies, ghrelin analogs, and ghrelin derivatives; and non-biologically active ghrelin analogues that compete with ghrelin for receptor binding. Additionally, the methods described herein comprise the use of ghrelin receptor antagonists, e.g., ghrelin receptor antibodies, ghrelin receptor peptides or fragments, non-peptide ghrelin receptor antagonists, ghrelin analogs, ghrelin derivatives (e.g., peptide fragments of ghrelin that bind specifically to the receptor, but do not induce the inflammatory response, e.g., a signal activity as would normally occur if ghrelin bound to the receptor), and small molecules. Any combination of ghrelin antagonist, ghrelin inhibitor, ghrelin receptor antagonist and/or ghrelin receptor inhibitor are encompassed by this invention.

Any form of intestinal inflammation can be treated with the methods of the present invention. Intestinal inflammation may be associated with, for example, any form of inflammatory diarrhea of the large and/or small bowel, including inflammatory bowel disease (e.g., ulcerative colitis, Crohn's disease), acute enterocolitis, autoimmune inflammation or chronic enterocolitis. The inflammation can be mediated by an agent such as a bacterium, a virus, a parasite or a toxin (e.g., a toxin produced by Clostridium difficile).

Additionally, any form of ghrelin-mediated inflammation can be treated by this invention. By “ghrelin-mediated inflammation” is meant inflammation associated with upregulation of ghrelin or ghrelin receptor.

Methods are also provided herein for treating inflammatory diarrhea, including inflammatory bowel disease (e.g., ulcerative colitis, Crohn's disease), and acute or chronic enterocolitis in a patient by inhibiting or modulating ghrelin activity, ghrelin binding to the ghrelin receptor or the signaling activity of the ghrelin receptor in the patient. In one embodiment, the methods comprise administering to the patient an effective amount of a ghrelin antagonist (e.g., a ghrelin antibody, ghrelin derivative or small molecule), a ghrelin inhibitor (e.g., a small molecule, a ghrelin receptor peptide or fragment or ghrelin antibody), a ghrelin receptor inhibitor (e.g., a small molecule, ghrelin receptor antibody, ghrelin analog or ghrelin derivative), or a non-biologically active ghrelin analogue that competes with ghrelin for receptor binding. In a second embodiment, the methods comprise administering to the patient an effective amount of a ghrelin receptor antagonist (e.g., a ghrelin receptor antibody, ghrelin receptor peptide or fragment, non-peptide ghrelin receptor antagonist, ghrelin analog, ghrelin derivative (e.g., peptide fragment of ghrelin that binds specifically to the receptor, but does not induce the inflammatory response, e.g., a signal activity as would normally occur if ghrelin bound to the receptor) or small molecule)

In another aspect, the invention features a composition for treating intestinal inflammation or ghrelin-mediated inflammation. Compositions comprise an agent that inhibits or modulates the activity of ghrelin or the ghrelin receptor and a pharmacologically or physiologically compatible carrier. The agent can act as a ghrelin or ghrelin receptor inhibitor or agonist. Agents specifically covered by the present invention are the aforementioned ghrelin inhibitors or modulators, such as ghrelin antagonists and ghrelin receptor antagonists. Such agents can be one or more of the following: ghrelin antibodies, ghrelin antagonists, non-biologically active ghrelin analogs (i.e., analogs that bind to the receptor but do not induce an inflammatory response), ghrelin receptor antagonists, ghrelin receptor antibodies, ghrelin receptor peptides or fragments, and non-peptide ghrelin receptor antagonists.

The present invention encompasses use of an inhibitor or antagonist of ghrelin or a ghrelin receptor for the manufacture of a medicament for use in the treatment of intestinal inflammation (gut inflammation). The invention also encompasses use of an inhibitor or antagonist of ghrelin or a ghrelin receptor for the manufacture of a medicament for use in the treatment of ghrelin-mediated inflammation. Inhibitors and antagonists are the aforementioned ghrelin inhibitors and antagonists and include ghrelin antibodies, ghrelin antagonists, non-biologically active ghrelin analogs that compete with ghrelin for receptor binding (i.e., analogs that bind to the receptor but do not induce an inflammatory response), ghrelin receptor antagonists, ghrelin receptor antibodies, ghrelin receptor peptides or fragments, and non-peptide ghrelin receptor antagonists.

The invention also provides for use of these medicaments in the treatment of patients with any form of inflammatory diarrhea of the large and/or small bowel, including inflammatory bowel disease (e.g., ulcerative colitis, Crohn's disease), or with acute and/or chronic enterocolitis.

In another aspect, the present invention also provides methods for identifying or screening for a ghrelin antagonist and/or a ghrelin receptor antagonist comprising (a) contacting cells expressing a ghrelin receptor with a candidate ghrelin antagonist or a candidate ghrelin receptor antagonist and with ghrelin; (b) determining MAP kinase phosphorylation in the cells which have been contacted with the candidate antagonist and with ghrelin; (c) comparing MAP kinase phosphorylation determined in step (b) with MAP kinase phosphorylation in control cells which have been contacted with ghrelin and which have not been contacted with the candidate antagonist; and (d) selecting the candidate antagonist if MAP kinase phosphorylation determined in step (b) is inhibited relative to MAP kinase phosphorylation in the control cells which have been contacted with ghrelin and which have not been contacted with the candidate antagonist, whereby the candidate antagonist is identified as a ghrelin antagonist or a ghrelin receptor antagonist. In a particular embodiment, cells expressing a ghrelin receptor are contacted with a candidate antagonist prior to contact with ghrelin. In another particular embodiment, steps (a) to (d) are repeated with a range of different concentrations of a candidate antagonist. The invention further relates to ghrelin antagonists and ghrelin receptor antagonists identified in accordance with the methods.

### BRIEF DESCRIPTION OF THE DRAWINGS

FIG. 1A is a bar graph representation showing the results on induction of ghrelin expression by TNBS treatment.

FIG. 1B is a bar graph representation showing the results on induction of ghrelin receptor (GHS-R) gene by TNBS treatment.

FIG. 2A is a bar graph representation showing the effect of activation of GHS-R on IL-8 promoter activity.

FIG. 2B is a bar graph representation showing the effect of ghrelin on IL-8 secretion.

FIG. 3A is a bar graph representation showing the effect of CAPE on ghrelin-induced IL-8 promoter activity.

FIG. 3B is a bar graph representation showing the effect of IκBαM overexpression on ghrelin-induced IL-8 promoter activity.

FIG. 4 is a bar graph representation showing the effect of the ghrelin receptor antagonist D-lys-GHRP-6 on C. difficile toxin A-induced secretion.

FIG. 5 shows the relative levels of GHS-R-1a mRNA in colons of patients with Crohn's disease (CD) or ulcerative colitis (UC) as compared to normal colons (noninflamed colons).

### DETAILED DESCRIPTION OF THE INVENTION

Communication between the endocrine and immune system is important in controlling the organism's response to inflammatory stimuli. Administration of growth hormone (GH) has been shown recently to ameliorate the symptoms of patients with inflammatory bowel disease (IBD) presumably by promoting mucosal healing (Slonim, A. E. et al., N. Engl. J. Med, 342:1633-1637 (2000)). The levels of circulating GH are controlled by at least two hypothalamic hormones: growth hormone releasing hormones (GHRH) that stimulate GH release, and somatostatin that inhibits GH release from the pituitary (Pombo, M. et al., Horm. Res., 55:11-16 (2001)). Ghrelin, a naturally occurring growth hormone (GH) secretotagoue, was identified by searching for an orphan G-protein coupled receptor which could be activated by a synthetic GH releasing peptide (Kojima, M. et al., Nature, 402:656-660 (1999)). Ghrelin is a unique acylated 28 amino-acid peptide, secreted predominantly from the stomach. Circulating ghrelin enters the central nervous system and stimulates GH release from the pituitary (Kojima, M. et al., Nature, 402:656-660 (1999)). Apart from the stomach, ghrelin and its receptor were also identified by RT-PCR technique in the small intestine as well as in the colon of animals and humans (Date, Y. et al., Endocrinology, 141:4255-4261 (2000); Hosoda, H. et al., Biochem. Biophys. Res. Commun., 279:909-913 (2000); Wang, G. et al., Regul. Pept., 105:75-81 (2002); Sakata, I. et al., Peptides, 23:531-536 (2002); Lee, H. M. et al., Endocrinology, 143:185-190 (2002)). Moreover, human endocrine tumors of the stomach and intestine also express ghrelin and its receptor (Papotti, M. et al., J. Clin. Endocrinol. Metab., 86:5052-5059 (2001)). Although evidence suggests that ghrelin-positive cells appear to be predominantly enteric neuroendocrine cells (Date, Y. et al., Endocrinology, 141:4255-4261 (2000); Lee, H. M. et al., Endocrinology, 143:185-190 (2002)), the precise cell types which express ghrelin and its receptor have not yet been localized.

Ghrelin Receptor and Signaling

Howard et al initially identified and cloned the receptor for ghrelin (GHS-R) by screening a swine pituitary cDNA library using a small synthetic hexapeptide (GHRP-6) that stimulates GH release by a pathway distinct from that of growth hormone releasing hormone (GHRH) (Howard, A. D. et al., Science, 273:974-977 (1996)). The ghrelin receptor belongs to a superfamily of seven-transmembrane domain-containing G protein-coupled receptors and has an open reading frame encoding a 353-amino acid protein. The same group also cloned the human full-length GHS-R (Howard, A. D. et al., Science, 273:974-977 (1996)). The rat cDNA was subsequently cloned and found to encode a protein of 364 amino acids containing seven transmembrane domains (7-TM) with 90% sequence identity to the human GHS-R. A single intron of approximately 2 kb divides the open reading frame into two exons encoding TM 1-5 and TM 6-7 (McKee, K. K. et al., Mol. Endocrinol., 11:415-423 (1997)). Recently, its natural ligand, ghrelin was found and purified from rat stomach using a stable CHO cell line expressing this rat GHS-R (Kojima, M. et al., Nature, 402:656-660 (1999)).

Prior to the cloning of GHS-R, its synthetic ligand GHRP-6 was shown to induce a transient increase in intracellular calcium in rat somatotrophes (Herrington, J. and Hille, B., Endocrinology, 135:1100-1108 (1994)), and stimulate phospholipase C and protein kinase C activity C (Cheng, K. et al., Endocrinology, 129:3337-3342 (1991); Mau, S. E. et al., J. Recept. Signal Transduct. Res., 15:311-323 (1995)). GHRP-6 had no effect on intracellular cAMP levels in rat primary pituitary cell culture though it potentiates the GRF-induced increase in cAMP levels (Cheng, K. et al., Endocrinology, 124:2791-2798 (1989)). Recently, ghrelin has been shown to activate MAP kinase and PI-3 kinase in the hepatoma cell line HepG2, and increase phosphorylation of insulin receptor substrate-1 (IRS-1), and the association of IRS-1 with GRB-2 and PI-3 kinases (Murata, M. et al., J. Biol. Chem., 277:5667-5674 (2002)). As described herein, ghrelin has now been shown to activate MAP kinase in colonocytes expressing ghrelin receptor.

Functions of Ghrelin

In addition to its potent GH-releasing activity, recent results indicate that ghrelin is a major regulator of food intake, energy production and body weight. Thus, ghrelin stimulates food intake and induces obesity, which appears to be independent of its GH-releasing activity (Wren, A. M. et al., Endocrinology, 141:4325-4328 (2000); Nakazato, M. et al., Nature, 409:194-198 (2001); Tschop, M. et al., Nature, 407:908-913 (2000)). Conversely, central administration of a ghrelin antibody inhibits appetite (Nakazato, M. et al., Nature, 409:194-198 (2001)). In humans, the plasma levels of ghrelin increase before each meal and decrease afterwards (Cummings, D. E. et al., N. Engl. J. Med., 346:1623-1630 (2002)). Fasting plasma ghrelin levels in anorectic patients are significantly higher and return to normal levels after partial weight recovery (Otto, B. et al., Eur. J. Endocrinol., 145:669-673 (2001)). Ghrelin levels are also elevated in Prader-Willi syndrome, a common form of human syndromic obesity (Cummings, D. E. et al., Nat. Med, 8:643-644 (2002)). The effect of ghrelin on food intake appears to be mediated by the neuropeptide Y since ghrelin can increase arcuate NPY expression, which in turn acts through Y1 receptors to increase food intake and decrease energy expenditure (Nakazato, M. et al., Nature, 409:194-198 (2001); Asakawa, A. et al., Gastroenterology, 120:337-345 (2001)). In the intestine, ghrelin stimulates gastric acid secretion and motility (Date, Y. et al., Biochem. Biophys. Res. Commun., 280:904-907 (2001); Masuda, Y. et al., Biochem. Biophys. Res. Commun., 276:905-908 (2000)), accelerates gastric emptying and small intestinal transit of a liquid meal (Trudel, L. et al., Am. J. Physiol. Gastrointest. Liver Physiol., 282:G948-G952 (2002)), while in the liver it stimulates proliferation of hepatoma cells (Murata, M. et al., J. Biol. Chem., 277:5667-5674 (2002)). Ghrelin is also implicated in the stress response since it induces potent anxiogenic activities (Asakawa, A. et al., Neuroendocrinology, 74:143-147 (2001)), while tail pinch stress and starvation stress induce ghrelin gene expression in the stomach (Asakawa, A. et al., Neuroendocrinology, 74:143-147 (2001)). The effects of ghrelin in stress appear to be mediated in part via corticotropin-releasing hormone (CRH) since administration of a CRH receptor antagonist significantly inhibits ghrelin-induced anxiogenic effects, and peripherally administered ghrelin significantly increases CRH mRNA in the hypothalamus (Asakawa, A. et al., Neuroendocrinology, 74:143-147 (2001)).

Gene Regulation of Ghrelin and its Receptor

Ghrelin gene expression in the stomach is increased by fasting and in leptin-deficient ob/ob mice. It is also negatively regulated by leptin, which suggests that leptin decreases food intake at least partially by inhibiting ghrelin expression (Asakawa, A. et al., Gastroenterology, 120:337-345 (2001)). However, the mechanisms by which starvation stress and leptin regulate ghrelin expression are not known. In addition, expression of ghrelin and its receptor is also developmentally regulated and GHRH infusion increases pituitary levels of both ghrelin and GHS-R mRNA (Kamegai, J. et al., Endocrinology, 142: 4154-4157 (2001)). However, little is known on the regulation of ghrelin and its receptor gene during other physiological and/or pathological states.

Ghrelin and Inflammation

Katugampola et al. reported that receptor density for ghrelin, which itself can cause vasodilatation, is elevated approximately 4-fold in the coronary artery of patients with coronary disease (Katugampola, S. D. et al., Clin. Sci. (Lond)., 103 Suppl 1:171S-175S (2002)). Interestingly, GH secretion increases in experimental arthritis, which is thought to be part of an adaptive process involved in the regulation of inflammation (Bluet-Pajot, M. T. et al., Neuroendocrinology, 63:85-92 (1996)). However, whether this response is linked to increased expression of ghrelin is not known.

As described herein, ghrelin and its receptor GHS-R have been shown to be significantly upregulated during colonic inflammation. The results described herein also indicate that ghrelin receptor is expressed in human colonic epithelial cells and that binding of ghrelin to this receptor in colonocytes induces (causes) release (expression) of the potent proinflammatory chemokine IL-8 via a mechanism involving activation of the transcription factor NF-κB. These novel discoveries indicate that ghrelin is involved in the pathophysiology of colonic inflammation. Since the transcription factor NF-κB is a global mediator of inflammatory responses leading to the activation of proinflammatory mediators, the results herein imply that ghrelin plays a role in the pathophysiology of all forms of inflammation where NF-κB is involved.

The expression of ghrelin and its receptor may be further characterized using experimental models of intestinal inflammation, such as the three models described herein. To determine whether interaction of ghrelin with its receptor plays a functional role in the pathophysiology of intestinal inflammation, different approaches may be used, including an anti-ghrelin antibody, a ghrelin receptor specific antagonist and ghrelin receptor knock out mice. Since recent studies suggest that substance P, neurotensin, CRH and calcitonin gene-related peptide (CGRP), by interacting with their respective receptors localized in the intestinal mucosa, play a proinflammatory role in C. difficile toxin A-mediated intestinal inflammation (Castagliuolo, I. et al., J. Clin. Invest., 103:843-849 (1999); Castagliuolo, I. et al., J. Clin. Invest., 101:1547-1550 (1998); Wlk, M. et al., Gastroenterology, 123:505-515 (2002); Pothoulakis, C. et al., Ann. N.Y. Acad. Sci., 840:635-648 (1998); Keates, A. C. et al., Am. J. Physiol., 274:G196-G202 (1998)), and TNBS and DSS-induced colitis (Stucchi, A. F. et al., Am. J. Physiol. Gastrointest. Liver Physiol., 279: G1298-G1306 (2000); Di Sebastiano, P. et al., Dig. Dis. Sci., 44:439-444 (1999)), the possibility that ghrelin mediates its intestinal responses by interacting with these peptides is also examined. Recent evidence also suggests that leptin plays a proinflammatory role in C. difficile toxin A enteritis (Mykoniatis, A. et al., “Leptin mediates Clostridium difficile-induced enteritis in mice”, Gastroenterology, 124(3):683-691 (2003); Wlk, M. et al., Gastroenterology, 123:505-515 (2002)) and TNBS and DSS-induced colitis (Siegmund, B. et al., Gastroenterology, 122:2011-2025 (2002); Barbier, M. et al., Life Sci., 69:567-580 (2001); Barbier, M. et al., Gut, 43:783-790 (1998)). These observations, combined with the results described herein, suggest that ghrelin and leptin may have similar proinflammatory effects despite their opposite effects on food intake and leptin inhibition of ghrelin expression in the stomach (Asakawa, A. et al., Gastroenterology, 120:337-345 (2001)).

Central Role of NF-κB in Inflammation

NF-κB is a transcription factor that is ubiquitously expressed and regulates the expression of numerous genes involved in the immune system and the inflammatory response (Silverman, N. and Maniatis, T., Genes Dev., 15:2321-2342 (2001); Ghosh, S. et al., Annu. Rev. Immunol., 16:225-260 (1998); Schmid, R. M. et al., Gut, 43:587-588 (1998); Ellis, R. D. et al., Inflamm. Res., 47:440-445 (1998); Ardite, E. et al., Br. J. Pharmacol., 124:431-433 (1998) 49-53)). It consists of homo- and heterodimers of Rel family proteins including p65 (RelA), RelB, c-rel, p50 and p52 response (Silverman, N. and Maniatis, T., Genes Dev., 15:2321-2342 (2001); Ghosh, S. et al., Annu. Rev. Immunol., 16:225-260 (1998)). The Rel family proteins share an N-terminal region of homology termed RHR (Rel Homology Region) that contains domains responsible for DNA binding, dimerization, and a nuclear localization signal (NLS) sequence. In unstimulated cells, NF-κB is sequestered in the cytoplasm by its inhibitory proteins called IκBs (Baeuerle, P. A. and Baltimore, D., Cell, 87:13-20 (1996); Baeuerle, P. A. and Baltimore, D., Science, 242:540-546 (1988)). Upon stimulation, a signal transduction cascade is initiated which leads to the phosphorylation of IκBs on two conserved serine residues at the N-terminus by the recently described IκB kinases (IKK). The phosphorylated IκBs are subsequently ubiquitinated on two conserved lysines at the N-terminus and degraded by the proteasome-mediated pathway (Peters, R. T. and Maniatis, T., Biochim. Biophys. Acta, 2:M57-M62 (2001); Karin, M. and Delhase, M., Semin. Immunol., 12:85-98 (2000)). Subsequently, NF-κB translocates into the nucleus and activates transcription of numerous proinflammatory genes. NF-κB/IκB pathway is critical for the expression of proinflammatory effects of several neuropeptides, such as neurotensin (Zhao, D. et al., J. Biol. Chem., 276:44464-44471 (2001)) and substance P (Lieb, K. et al., J. Immunol., 159:4952-4958 (1997)).

As described herein, ghrelin-induced IL-8 gene expression has been shown to be inhibited by CAPE, a specific inhibitor of the NF-κB pathway, and by expression of a superrepressor IκBαM. These surprising and unexpected discoveries indicate that the NF-κB pathway plays a role in ghrelin-induced IL-8 expression. Experiments, as described herein, can be conducted to further characterize the role of the NF-κB pathway in ghrelin-induced IL-8 expression and to determine the upstream signaling pathways leading to this response.

Inflammatory Bowel Disease (IBD)

Inflammatory Bowel Disease (IBD) is a chronic debilitating recurrent inflammatory disease that affects either or both the small intestine and the colon. IBD comprises two major groups: Crohn's disease and ulcerative colitis. IBD exhibits a clinical course characterized by successive exacerbation and remissions (Fiocchi, C., Gastroenterology, 115:182-205 (1998)). IBD affects millions of patients per year worldwide (McFarland, L. V. et al., N. Engl. J. Med, 320:204-210 (1989)), has poor prognosis and: its treatment is primarily symptomatic. C. difficile toxin-associated colitis represents the major cause of infectious colitis in hospitals in the United States (Kelly, C. P. et al., N. Engl. J. Med., 330:257-262 (1994)), affecting millions of patients per year worldwide (McFarland, L. V. et al., N. Engl. J. Med., 320:204-210 (1989)). Moreover, despite their significant morbidity and mortality, the pathophysiology of IBD, and the precise mechanisms involved in C. difficile toxin-mediated colitis, are not completely understood. Evidence suggests that inflammatory mediators amplify the inflammatory process and produce mucosal dysfunction. During the past few years, the pathophysiology of intestinal inflammation has evolved to a model that includes interactions between epithelial cells, brain-gut peptide/hormones and immune and inflammatory cells of the intestinal mucosa.

In addition to the well-accepted role of bacteria, immune cells, and pro-inflammatory cytokines, such as TNFα and IL-1β, neuro-immune interactions have been recently demonstrated to play an important role in the pathophysiology of intestinal inflammation. For example, a dramatic upregulation of neuropeptide expression, such as neurotensin, substance P, corticotropin-releasing hormone (CRH), calcitonin gene-releasing peptide (CGRP) and their receptors, is evident during intestinal inflammation (Castagliuolo, I. et al., J. Clin. Invest., 103:843-849 (1999); Castagliuolo, I. et al., J. Clin. Invest., 101:1547-1550 (1998); Wlk, M. et al., Gastroenterology, 123:505-515 (2002); Pothoulakis, C. et al., Ann. N.Y. Acad. Sci., 840:635-648 (1998); Keates, A. C. et al., Am. J. Physiol., 274:G196-G202 (1998). Furthermore, receptor blockade significantly inhibits intestinal inflammation (Castagliuolo, I. et al., J. Clin. Invest., 103:843-849 (1999); Castagliuolo, I. et al., J. Clin. Invest., 101:1547-1550 (1998); Wlk, M. et al., Gastroenterology, 123:505-515 (2002); Pothoulakis, C. et al., Ann. N.Y. Acad. Sci., 840:635-648 (1998); Keates, A. C. et al., Am. J. Physiol., 274:G196-G202 (1998)). Leptin, a major hormone/peptide known to lower appetite and control metabolic responses, is also implicated in the pathophysiology of colitis (Siegmund, B. et al., Gastroenterology, 122:2011-2025 (2002)). Crohn's disease is characterized by inflammation and ulceration occurring deep in the intestinal wall layers. The lower part of the small intestine, the ileum, is one of the most common areas affected. Infrequently, the disease can affect any portion of the gastrointestinal tract. Symptoms include abdominal pain, frequently localized in the lower right side, diarrhea, and weight loss, as well as rectal bleeding and fever. A common complication of Crohn's disease is intestinal blockage or stricture. This occurs as a result of the disease's thickening of the intestinal walls. Fistulas, which frequently occur around the anus and rectum, are another common complication of the disease. Fistulas are abnormal openings that result when ulcers in the intestine create passageways into the surrounding tissues of the bladder, vagina or skin.

Ulcerative colitis affects the colon and typically gives rise to diarrhea, abdominal

cramps and rectal bleeding. It may also be accompanied by fatigue, weight loss, appetite loss, loss of body fluids and nutrients, and abdominal pain. A salient feature of ulcerative colitis is that the inflammation of the colon is uniform and continuous. The disease may be limited to the rectum (known as proctitis), may involve part of the colon, or may involve the entire colon. The surface mucosal cells, including the submucosa and the crypt epithelium, play a role in the inflammatory reaction. As disease progresses, epithelial damage ensues along with loss of surface epithelial cells. This gives rise to multiple ulcerations. Accordingly, about 85% of patients with ulcerative colitis have mild to moderate disease which can be managed without hospitalization. In the remaining 15%, the patients' entire colon are involved and the disease is accompanied by severe bloody diarrhea and systemic symptoms. Toxic dilation of the colon is common among patients with severe ulcerative colitis.

Experimental Models of Colitis

Participation of neuro-immune interactions in intestinal inflammation has been demonstrated in three models of colitis: TNBS-induced colitis, DSS-induced colitis and C. difficile toxin A-induced colitis. For example, using models of acute and chronic intestinal inflammation, substance P, CGRP, CRH and neurotensin have been shown to promote inflammation by interacting with their receptors in the intestinal mucosa. Substance P has been shown to be required for both TNBS-induced colitis (Mazelin, L. et al., Life Sci., 63:293-304 (1998)) and toxin A-induced colitis (Castagliuolo, I. et al., J. Clin. Invest., 101:1547-1550 (1998)). Other neuropeptides may play a different role in different models of colitis. For example CGRP has been shown to inhibit TNBS colitis (Fiorucci, S. et al., Proc. Natl. Acad. Sci. USA, 98:13936-13941 (2001); Mazelin, L. et al., Peptides, 20:1367-1374 (1999)), but plays a proinflammatory role in toxin A-induced enteritis (Keates, A. C. et al., Am. J. Physiol., 274:G196-G202 (1998)).

TNBS Model of Colitis

The TNBS model of colitis has been commonly used to study the athophysiologic mechanisms of formation of intestinal inflammation. TNBS colitis is a lymphocyte dependent model induced by intracolonic administration of 2,4,6-trinitrobenzene sulfonic acid (TNBS). Rectal administration of low doses of TNBS induces a chronic colitis with several features similar to those in Crohn's disease in humans. These include severe diarrhea, weight loss, and rectal prolapse, the presence of a wasting syndrome as well as histologic features of Crohn's disease, such as granuloma formation and mucosal infiltration of neutrophils (Fiocchi, C., Gastroenterology, 115:182-205 (1998)). The TNB S model is a TH-1-driven colitis and depends largely on the production of IL-12 (Bouma, G. et al., Gastroenterology, 123:554-565 (2002); Neurath, M. F. et al., J. Exp. Med., 182:1281-1290 (1995)).

DSS Model of Colitis

The DSS model of colitis has also been commonly used to study the pathophysiologic mechanisms of formation of intestinal inflammation. DSS colitis is a lymphocyte-independent model generated by oral administration of dextran sodium sulfate (DSS) (Dieleman, L. A. et al., Gastroenterology, 107:1643-1652 (1994)). Colitis in response to DSS administration appears to involve primarily bacterial translocation and macrophage activation (Dieleman, L. A. et al., Gastroenterology, 107:1643-1652 (1994)).

C. difficile Toxin A-Induced Enteritis

C. difficile toxin A-induced enteritis is a model for studying acute enterotoxin-mediated diarrhea and inflammation and is linked to the pathophysiology of antibiotic-associated diarrhea and colitis in humans (Pothoulakis, C., Ann. N. Acad. Sci., 915:347-356 (2000)). C. difficile induces colitis by releasing two exotoxins: toxin A and toxin B. Both toxins cause damage of intestinal mucosa, increase intestinal permeability, intramural fluid secretion and neutrophil infiltration.

The results herein indicate that ghrelin may be directly involved in inflammatory responses in the gut by activating nuclear translocation of the transcription factor κB and stimulating secretion of proinflammatory cytokines. The results herein also show that ghrelin and ghrelin receptor mRNA are upregulated in the colon in the acute phase of experimental colitis. The results herein further show that ghrelin receptor mRNA is upregulated in the colons of human patients with Crohn's disease or ulcerative colitis. These results elucidate a role for ghrelin and its receptor in the pathophysiology of gut or colonic inflammation. The results eluciate a role for ghrelin and its receptor in the pathophysiology of enteritis and intestinal inflammation. Since the transcription factor NF-κB is a global mediator of inflammatory responses leading to the activation of proinflammatory mediators, the results imply that ghrelin and its receptor play a role in the pathophysiology of all forms of inflammation where NF-κB is involved.

Experiments can be conducted to examine the localization of ghrelin and its colonic receptors as described herein. Studies on their modulation during colonic inflammation in experimental colitis can also be conducted as described herein. Ghrelin, similar to other neuropeptides, may represent a link between the endocrine and immune system during colonic inflammation. The studies described herein can be used to identify the pathway(s) by which ghrelin participates in experimental colonic inflammation and provide novel insights on the mechanism(s) by which this peptide modulates colonic inflammatory responses. These studies lead to novel targets for new therapeutic approaches for IBD, particularly antagonism of ghrelin and its receptor.

The present invention provides for use of an inhibitor or antagonist of ghrelin or a ghrelin receptor for the manufacture of a medicament for use in the treatment of intestinal inflammation (gut inflammation). The invention also encompasses use of an inhibitor or antagonist of ghrelin or a ghrelin receptor for the manufacture of a medicament for use in the treatment of ghrelin-mediated inflammation. By “ghrelin-mediated inflammation” is meant inflammation associated with upregulation of ghrelin or ghrelin receptor. Inhibitors and antagonists include ghrelin antibodies, ghrelin antagonists, non-biologically active ghrelin analogs that compete with ghrelin for receptor binding (i.e., analogs that bind to the receptor but do not induce an inflammatory response), ghrelin receptor antagonists, ghrelin receptor antibodies, ghrelin receptor peptides or fragments, and non-peptide ghrelin receptor antagonists.

The invention encompasses the use of these medicaments in the treatment of any form of intestinal inflammation. Intestinal inflammation may be associated with or cause by, for example, any inflammatory response, such as an autoimmune response, a parasitic infection, a response associated with a disease, such as IBD (e.g., ulcerative colitis, Crohn's disease), acute enterocolitis or chronic enterocolitis, or any form of inflammatory diarrhea of the large and/or small bowel, including that associated with IBD (e.g., ulcerative colitis, Crohn's disease), acute enterocolitis and chronic enterocolitis. The inflammation can be mediated by an agent such as a bacterium (e.g., Clostridium difficile), a virus, a parasite or a toxin (e.g., TxA toxin produced by Clostridium difficile). Intestinal inflammation occurs in intestinal tissues (e.g., the small or large intestine, ileum or colon).

The invention also provides for use of these medicaments in the treatment of patients with any form of inflammatory diarrhea of the large and/or small bowel, including inflammatory bowel disease (e.g., ulcerative colitis, Crohn's disease), or with acute and/or chronic enterocolitis either from bacterial, viral or toxin-mediated etiology.

The present invention encompasses methods of inhibiting or decreasing an inflammatory response in intestinal tissue comprising administering an effective amount of an agent such as, for example, a ghrelin or ghrelin receptor antagonist, thereby inhibiting the inflammatory response. The methods of the present invention encompass methods of inhibiting or decreasing intestinal inflammation in a mammal by administering an effective amount of an agent such as, for example, a ghrelin or ghrelin receptor antagonist, to inhibit or decrease intestinal inflammation. Administering the agent can be by means of directly contacting intestinal tissue cells with the agent or by delivering the agent alone or in a composition with an acceptable carrier or delivery vehicle. Methods are known in the art to contact or deliver an agent to a target tissue or tissue-specific cells (e.g., epithelial and lamina propria cells).

The present invention encompasses methods of treating intestinal inflammation (gut inflammation) comprising inhibiting or modulating ghrelin activity, ghrelin binding to the ghrelin receptor or the signaling activity of the ghrelin receptor. The present invention also encompasses methods for treating ghrelin-mediated inflammation comprising inhibiting or modulating ghrelin activity, ghrelin binding to the ghrelin receptor or the signaling activity of the ghrelin receptor. The methods disclosed herein contemplate the use of an agent that inhibits, i.e., inhibitors or antagonists, or modulates, e.g., agonists or other effectors, the activity of ghrelin or the ghrelin receptor such that inflammation is reduced or inhibited. An agent can be any molecule, chemical or biological, that modulates the activity of ghrelin or the ghrelin receptor. In particular, the methods disclosed herein comprise the use of ghrelin antagonists, e.g., ghrelin antibodies, ghrelin derivatives, and small molecules; ghrelin inhibitors, e.g., small molecules, ghrelin receptor peptides or fragments, and ghrelin antibodies; ghrelin receptor inhibitors, e.g., small molecules, ghrelin receptor antibodies, ghrelin analogs, and ghrelin derivatives; and non-biologically active ghrelin analogues that compete with ghrelin for receptor binding. Additionally, the methods described herein comprise the use of ghrelin receptor antagonists, e.g., ghrelin receptor antibodies, ghrelin receptor peptides or fragments, non-peptide ghrelin receptor antagonists, ghrelin analogs, ghrelin derivatives (e.g., peptide fragments of ghrelin that bind specifically to the receptor, but do not induce the inflammatory response, e.g., a signal activity as would normally occur if ghrelin bound to the receptor), and small molecules. Any combination of ghrelin antagonist, ghrelin inhibitor, ghrelin receptor antagonist and/or ghrelin receptor inhibitor are encompassed by this invention. Any form of intestinal inflammation can be treated with the methods of the present invention. Additionally, any form of ghrelin-mediated inflammation can be treated by this invention.

Methods are also provided herein for treating inflammatory diarrhea, including inflammatory bowel disease, such as, but not limited to, ulcerative colitis and Crohn's disease, and acute or chronic enterocolitis in a patient by inhibiting or modulating ghrelin activity, ghrelin binding to the ghrelin receptor or the signaling activity of the ghrelin receptor in the patient. In one embodiment, the methods comprise administering to the patient an effective amount of a ghrelin antagonist (e.g., a ghrelin antibody, ghrelin derivative or small molecule), a ghrelin inhibitor (e.g., a small molecule, a ghrelin receptor peptide or fragment or ghrelin antibody), a ghrelin receptor inhibitor (e.g., a small molecule, ghrelin receptor antibody, ghrelin analog or ghrelin derivative), or a non-biologically active ghrelin analogue that competes with ghrelin for receptor binding. In a second embodiment, the methods comprise administering to the patient an effective amount of a ghrelin receptor antagonist (e.g., a ghrelin receptor antibody, ghrelin receptor peptide or fragment, non-peptide ghrelin receptor antagonist, ghrelin analog, ghrelin derivative (e.g., peptide fragment of ghrelin that binds specifically to the receptor, but does not induce the inflammatory response, e.g., a signal activity as would normally occur if ghrelin bound to the receptor) or small molecule)

The invention encompasses modulation of ghrelin activity or ghrelin receptor activity in vertebrates, particularly in mammals. The methods of the invention are suitable for veterinary use as well as for treating humans. For example, canines exposed to toxins that result in ghrelin-mediated intestinal inflammation can be treated using the methods and/or agents described herein. The term “patient” is meant to encompass human patients and non-human patients.

An agent “modulates” activity if it alters the activity from that which would be exhibited in the absence of the agent. For example, inhibitors decrease activity, e.g., functional inhibitors that interact and block an active site, or competitive inhibitors that compete for binding; antagonists inhibit binding activity, e.g., molecules that reduce binding affinity between a receptor and ligand; and agonists increase binding activity, e.g., molecules that increase binding affinity between a receptor and a ligand. Examples of such molecules include, but are not limited to, ghrelin antibodies, small molecule agents, ghrelin agonists, ghrelin antagonists, non-biologically active ghrelin analogs, ghrelin receptors, ghrelin receptor agonists and ghrelin receptor antagonists. These agents can be proteins, peptides, peptide analogs, or chemical compounds or derivatives.

As used herein, ghrelin antagonists, such as ghrelin antibodies, block, interfere with, decrease or abrogate ghrelin function and/or biological activity in vivo. By “ghrelin function” is meant the biological function or action of ghrelin.

Ghrelin antibodies and ghrelin antagonists inhibit the activity of ghrelin and the binding of ghrelin to its receptor, thereby mitigating intestinal inflammation and/or ghrelin-mediated inflammation. Ghrelin antibodies are also referred to herein as anti-ghrelin antibodies and can specifically bind to the ghrelin receptor, thus preventing ghrelin binding to the receptor, and thereby, inhibiting or decreasing ghrelin receptor signaling and the resulting inflammatory response. Ghrelin antagonists are known and readily available. For example, ghrelin antibodies are commercially available. As discussed further herein, ghrelin antibodies can also be readily made using methods known and readily available in the art. Ghrelin antagonists can also be identified in accordance with the methods described herein. Ghrelin antagonists and antibodies can be combined with pharmaceutically-acceptable compositions and carriers to form compositions.

As used herein, ghrelin receptor antagonists, such as ghrelin receptor antibodies, ghrelin receptor peptides and non-peptide ghrelin receptor antagonists, block, interfere with, decrease or abrogate ghrelin receptor function and/or biological activity in vivo. By “ghrelin receptor function” is meant the biological function or action of ghrelin receptor. Examples of ghrelin receptor antagonists include ghrelin receptor antibodies and [D-lys-3]-GHRP-6. Ghrelin receptor antibodies can be readily made using methods known and readily available in the art. Ghrelin receptor antibodies are also commercially available. The ghrelin receptor antagonist [D-lys-3]-GHRP-6 is a hexapeptide (His-D-Trp-D-Lys-Trp-D-Phe-Lys-NH₂) that is commercially available.

The ghrelin receptor antagonist [D-lys-3]-GHRP-6 has also been described in the art (see, e.g., Smith, R. G. et al., Science, 260:1640-1643 (1993)). Other ghrelin receptor antagonists are known and readily available. Ghrelin receptor antagonists can also be identified in accordance with the methods described herein. Ghrelin receptor antagonists can be combined with pharmaceutically-acceptable compositions and carriers to form compositions.

Antibodies can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody”, as used herein, also encompasses functional fragments of antibodies, including fragments of human, chimeric, humanized, primatized, veneered or single chain antibodies. Functional fragments include antigen-binding fragments specific for ghrelin or ghrelin receptor. Antigen-binding fragments specific for ghrelin or ghrelin receptor include, but are not limited to, Fab, Fab′, F(ab′)₂ and Fv fragments. Such fragments can be produced by enzymatic cleavage or recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

Single chain antibodies, and chimeric, humanized or primatized (CDR-grafted), or veneered antibodies, as well as chimeric, CDR-grafted or veneered single chain antibodies, comprising portions derived from different species, and the like are also encompassed by the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0 125 023 B1; Boss et al., U.S. Pat. No. 4,816,397; Boss et al., European Patent No. 0 120 694 B1; Neuberger et al., International Publication No. WO86/01533; Neuberger et al., European Patent No. 0 194 276 B1; Winter et al., U.S. Pat. No. 5,225,539; Winter et al., European Patent No. 0 239 400 B1; Queen et al., European Patent No. 0 451 216 B1; and Padlan et al., EP 0 519 596 A1. See also, Newman et al., BioTechnology, 10:1455-1460 (1992), regarding primatized antibody, and Ladner et al., U.S. Pat. No. 4,946,778 and Bird et al., Science, 242:423-426 (1988)) regarding single chain antibodies. Antibodies for ghrelin and ghrelin receptor are also commercially available.

An “antigen” is a molecule or a portion of a molecule capable of being bound by an antibody which is additionally capable of inducing an animal to produce antibody capable of binding to an epitope of that antigen. An antigen can have one or more than one epitope.

The term “epitope” is meant to refer to that portion of the antigen capable of being recognized by and bound by an antibody at one or more of the antibody's antigen binding region. Epitopes usually consist of chemically active surface groupings of molecules such as amino acids or sugar side chains and have specific three dimensional structural characteristics as well as specific charge characteristics.

As used herein, the term “specific” when referring to an antibody-antigen interaction, is used to indicate that the antibody can selectively bind to ghrelin (in the case of a ghrelin antibody) or ghrelin receptor (in the case of a ghrelin receptor antibody).

Ghrelin, ghrelin receptor, or antigenic epitopes of ghrelin or the ghrelin receptor can be used to generate antibodies that are specific for ghrelin or its receptor.

The present invention includes the use of agents that are ghrelin analogs or derivatives of either ghrelin or the ghrelin receptor. Analogs, as used herein, are molecules that are structurally similar to, for example, ghrelin, and act to compete with ghrelin for ghrelin receptor binding sites. Non-biologically active ghrelin analogues (analogs) that compete with ghrelin for receptor binding, as used herein, are proteins or peptides that bind to the receptor but do not induce a ghrelin-mediated inflammatory response. Ghrelin or ghrelin receptor or derivatives, as used herein, are peptides or proteins having amino acid sequences analogous to endogenous ghrelin or the ghrelin receptor. Ghrelin derivatives can be used, for example, as a competitive inhibitor of ghrelin binding by competing for ghrelin receptor binding sites. The present invention includes the use of such ghrelin derivatives that are able to bind to the ghrelin receptor, but do not induce a ghrelin-mediated inflammatory response. Ghrelin receptor derivatives can be used, for example, to sequester unbound ghrelin, thereby reducing the ghrelin levels available to bind and induce endogenous ghrelin receptors. Analogous amino acid sequences are defined herein to mean amino acid sequences with sufficient identity of amino acid sequence of endogenous ghrelin to possess the biological activity of endogenous ghrelin or a slightly altered activity, e.g., reduced ghrelin receptor binding affinity, as well as analogous proteins that exhibit greater, or lesser activity than endogenous ghrelin. The derivatives or analogs of the present invention can also be “peptide mimetics,” peptides or proteins that contain chemically modified or non-naturally occurring amino acids. These mimetics can be designed and produced by techniques known to those of skill in the art (see, e.g., U.S. Pat. Nos. 4,612,132; 5,643,873 and 5,654,276, the teachings of which are herein incorporated by reference).

Systematic substitution of amino acids within the ghrelin protein can also be used to engineer high-affinity protein agonists and antagonists to the ghrelin receptor. Accordingly, the engineered ghrelin would exhibit enhanced or diminished affinity for binding with the ghrelin receptor. Such agonists and antagonists can be used to suppress or modulate the activity of ghrelin, thereby mitigating diarrhea, intestinal inflammation of ghrelin-mediated inflammation. Antagonists to ghrelin are applied in situations of gut inflammation, to block the inhibitory effects of ghrelin and mitigate the inflammation.

Candidate ghrelin receptor inhibitors or antagonists can also be identified by evaluating the binding of ghrelin to its receptor in the presence and absence of the candidate inhibitor antagonist. Such techniques are well-known to those of skill in the art. Alternatively, candidate ghrelin receptor inhibitors or antagonists can be identified by measuring ghrelin receptor signaling activity by the methods described herein (e.g., determining MAP kinase phosphorylation).

The present invention also encompasses methods for identifying or screening for a ghrelin antagonist and/or a ghrelin receptor antagonist comprising (a) contacting cells expressing a ghrelin receptor with a candidate ghrelin antagonist or a candidate ghrelin receptor antagonist and with ghrelin; (b) determining MAP kinase phosphorylation in the cells which have been contacted with the candidate antagonist and with ghrelin; (c) comparing MAP kinase phosphorylation determined in step (b) with MAP kinase phosphorylation in control cells which have been contacted with ghrelin and which have not been contacted with the candidate antagonist; and (d) selecting the candidate antagonist if MAP kinase phosphorylation determined in step (b) is inhibited relative to MAP kinase phosphorylation in the control cells which have been contacted with ghrelin and which have not been contacted with the candidate antagonist, whereby the candidate antagonist is identified as a ghrelin antagonist or a ghrelin receptor antagonist. In a particular embodiment, cells are contacted with a candidate antagonist prior to contact with ghrelin. In another particular embodiment, steps (a) to (d) are repeated with a range of different concentrations of a candidate antagonist.

By “range of different concentrations of the candidate antagonist” is meant 2 or more (i.e., 2, 3, 4, 5, etc.) different concentrations of the candidate antagonist.

Selecting concentration ranges is well within the ability of those skilled in the art.

By “cells expressing a ghrelin receptor” is meant cells or cell lines which express an endogenous ghrelin receptor or cells manufactured to express a ghrelin receptor or ghrelin receptor fragment. Cells expressing a ghrelin receptor or ghrelin receptor fragment can be manufactured by introducing into cells a DNA construct comprising a vector and a ghrelin receptor gene operably linked to a promoter. DNA constructs can be introduced into cells according to methods known in the art (e.g., transformation, direct uptake, calcium phosphate precipitation, electroporation, projectile bombardment, using liposomes). Such methods are described in more detail, for example, in Sambrook et al., Molecular Cloning: A Laboratory Manual, 3rd edition (New York: Cold Spring Harbor University Press) (2001); and Ausubel et al., Current Protocols in Molecular Biology (New York: John Wiley & Sons) (1998).

A vector, as the term is used herein, refers to a nucleic acid vector, e.g., a DNA plasmid, virus or other suitable replicon (e.g., viral vector). Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, 3rd Edition, B.N. Fields, et al., eds., Philadelphia, Pa.: Lippincott-Raven Publishers) (1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., U.S. Pat. No. 5,801,030, the teachings of which are incorporated herein by reference.

A nucleic acid sequence encoding a protein or peptide (e.g., ghrelin receptor, ghrelin receptor fragment) can be inserted into a nucleic acid vector according to methods generally known in the art (see, e.g., Ausubel et al., eds., Current Protocols in Molecular Biology, John Wiley & Sons, Inc., New York (1998); Sambrook et al., Molecular Cloning. A Laboratory Manual, 3rd edition (New York: Cold Spring Harbor University Press) (2001)).

A nucleic acid sequence encoding a ghrelin receptor or ghrelin receptor fragment can be isolated from nature, modified from native sequences or manufactured de novo, as described in, for example, Ausubel et al., Current Protocols in Molecular Biology, (New York: John Wiley & Sons) (1998); and

Sambrook et al., Molecular Cloning: A Laboratory Manual, 3rd edition (New York: Cold Spring Harbor University Press) (2001). Nucleic acids can be isolated and fused together by methods known in the art, such as exploiting and manufacturing compatible cloning or restriction sites.

Typically, the nucleic acid sequence will be a gene which encodes the desired ghrelin receptor or ghrelin receptor fragment. Such a gene is typically operably linked to suitable control sequences capable of effecting the expression of the ghrelin receptor ghrelin receptor fragment. The term “operably linked”, as used herein, is defined to mean that the gene (or the nucleic acid sequence) is linked to control sequences in a manner which allows expression of the gene (or the nucleic acid sequence). Generally, operably linked means contiguous.

Control sequences include a transcriptional promoter, an optional operator sequence to control transcription, a sequence encoding suitable messenger RNA (mRNA) ribosomal binding sites and sequences which control termination of transcription and translation. In a particular embodiment, a recombinant gene (or a nucleic acid sequence) encoding a ghrelin receptor or ghrelin receptor can be placed under the regulatory control of a promoter, which can be induced or repressed, thereby offering a greater degree of control with respect to the level of the product.

As used herein, the term “promoter” refers to a sequence of DNA, usually upstream (5′) of the coding region of a structural gene, which controls the expression of the coding region by providing recognition and binding sites for RNA polymerase and other factors which may be required for initiation of transcription. Suitable promoters are well known and readily available in the art (see, e.g., Ausubel et al., Current Protocols in Molecular Biology (New York: John Wiley & Sons, Inc.) (1998); Sambrook et al, Molecular Cloning: A Laboratory Manual, 3rd edition (New York: Cold Spring Harbor University Press) (2001); and U.S. Pat. No. 5,681,735).

Cells contacted with a candidate antagonist and/or ghrelin will take up the candidate antagonist and/or ghrelin.

As used herein, a cell refers to an animal cell. The cell can be a stem cell or somatic cell. Suitable animal cells can be of, for example, mammalian origin. Examples of mammalian cells include human (such as HepG2 cells, HeLa cells), bovine, ovine, porcine, rodent (such as rat (such as intestinal epithelial cells (e.g., IEC-6 cells)), murine (such as embryonic stem cells), rabbit etc.) and monkey (such as COS1 cells) cells. Preferably, the cell is of intestinal origin (such as intestinal epithelial cells, etc.). The cell can also be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell, or pathogen-infected cell (e.g., those infected by bacteria, viruses, virusoids, parasites, or prions). The cells can be obtained commercially or from a depository or obtained directly from an animal, such as by biopsy.

Candidate ghrelin antagonists and candidate ghrelin receptor antagonists can be individually screened or one or more candidate antagonist(s) can be tested simultaneously in accordance with the methods herein. Candidate antagonists include organic compounds, chemical compounds, ionic compounds, organic ligands, including cofactors, saccharides, recombinant and synthetic peptides, proteins, including antibodies, peptoids, nucleic acid sequences, including genes, nucleic acid products, pharmaceutical agents, drugs, and other molecules and compositions. Where a mixture of candidate antagonists is tested, the antagonists selected by the methods described can be separated (as appropriate) and identified by suitable methods (e.g., chromatography, sequencing, PCR). The presence of one or more ghrelin antagonists and/or ghrelin receptor antagonists in a test sample can also be determined according to these methods.

Large combinatorial libraries of compounds (e.g., organic compounds, recombinant or synthetic peptides, peptoids, nucleic acids) produced by combinatorial chemical synthesis or other methods can be tested (see e.g., Zuckerman, R. N. et al., J. Med. Chem., 37:2678-2685 (1994) and references cited therein; see also, Ohlmeyer, M. H. J. et al., Proc. Natl. Acad. Sci. USA, 90:10922-10926 (1993) and DeWitt, S. H. et al., Proc. Natl. Acad. Sci. USA, 90:6909-6913 (1993), relating to tagged compounds; Rutter, W. J. et al. U.S. Pat. No. 5,010,175; Huebner, V. D. et al., U.S. Pat. No. 5,182,366; and Geysen, H. M., U.S. Pat. No. 4,833,092). The teachings of these references are incorporated herein by reference. Where compounds selected from a combinatorial library carry unique tags, identification of individual compounds by chromatographic methods is possible.

Chemical libraries, microbial broths and phage display libraries can also be tested (screened) for the presence of one or more ghrelin antagonist(s) or ghrelin receptor antagonist(s) in accordance with the methods herein.

MAP kinase phosphorylation is determined using methods generally known in the art (e.g., Western blot analysis using phospho-MAPK specific antibody).

Such methods are described in more detail, for example, in Sambrook et al., Molecular Cloning: A Laboratory Manual, 3rd edition (New York: Cold Spring Harbor University Press) (2001); and Ausubel, et al., Current Protocols in Molecular Biology (New York: John Wiley & Sons) (1998).

The present invention further encompasses cells expressing a ghrelin receptor produced in accordance with the methods herein and ghrelin antagonists and ghrelin receptor antagonists identified in accordance with the methods herein.

Ghrelin antagonists and ghrelin receptor antagonists identified in accordance with the screening methods herein can be administered to a patient in accordance with the methods herein.

Agents of the present invention (i.e., the aforementioned ghrelin inhibitors or modulators, such as ghrelin antagonists and ghrelin receptor antagonists) can be administered alone (naked administration) or as part of a composition. Routes of administration are generally known in the art and include aerosol, oral, systematic, intravenous including infusion and/or bolus injection, intrathecal, parenteral, mucosal, implant, intraperitoneal, intradermal, transdermal (e.g., in slow release polymers), intramuscular, subcutaneous, topical, epidural, etc. routes. Other suitable routes of administration can also be used, for example, to achieve absorption through epithelial or mucocutaneous linings. Agents of the present invention can also be administered by gene therapy, wherein a DNA molecule encoding a particular therapeutic protein or peptide is administered to the patient, e.g., via a vector, which causes the particular protein or peptide to be expressed and secreted at therapeutic levels in vivo.

Agents and compositions of the present invention can be administered together with other components of biologically active agents, such as pharmaceutically acceptable surfactants (e.g., glycerides), excipients (e.g., lactose), carriers, diluents and vehicles. If desired, certain sweetening, flavoring and/or coloring agents can also be added.

Agents and compositions of the present invention can be administered prophylactically or therapeutically to an individual prior to, simultaneously with or sequentially with other therapeutic regimens or agents (e.g., multiple drug regimens), including with other therapeutic regimens used for the treatment of inflammation, including gut inflammation and related disorders. Agents or compositions that are administered simultaneously with other therapeutic agents can be administered in the same or different compositions.

It may be undesirable to administer the protein systemically because of side-affects. To eliminate pleiotropic effects of administering an agent included in the present invention, it would be useful to deliver (or target) the agent to a specific tissue (e.g., intestinal tissue or ghrelin receptor positive epithelial or lamina propria cells). One way to deliver the agent to a specific tissue is to conjugate the protein with a targeting agent. For example, the protein can comprise a peptide to target the ghrelin receptor to a specific tissue or cell type, e.g., intestinal tissue or cells. Such targeting molecules are well known to those of skill in the art.

Agents and compositions of the present invention can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation can be sterilized by commonly used techniques. Suitable pharmaceutical carriers are described in Remington's Pharmaceutical Sciences.

The term “pharmaceutically acceptable” can be used interchangeably with “physiologically acceptable” to mean a non-toxic material that does not interfere with the effectiveness of the biological activity of the active ingredient(s). An “effective amount” of agent or composition is defined herein as that amount, or dose, of agent or composition of the invention that, when administered to the subject, is sufficient to reduce or inhibit intestinal inflammation or ghrelin-mediated inflammation in a specific tissue or cell. The dosage administered to a subject will vary depending upon a variety of factors, including the pharmacodynamic characteristics of the particular agent or composition, and its mode and route of administration; size, age, sex, health, body weight and diet of the recipient; nature and extent of symptoms of the disease or condition being treated, kind of concurrent treatment, frequency of treatment, and the effect desired.

An effective amount can be administered in single or divided doses (e.g., a series of doses separated by intervals of days, weeks or months), or in a sustained release form, depending upon factors such as nature and extent of symptoms, kind of concurrent treatment and the effect desired. Other therapeutic regimens or agents can be used in conjunction the present invention. Adjustment and manipulation of established dosage ranges are well within the ability of those skilled in the art.

Once an effective amount has been administered, a maintenance amount of an agent or composition of the invention can be administered to the subject. A maintenance amount is the amount of agent or necessary to maintain the reduction or inhibition of the inflammatory response mediated by ghrelin and ghrelin receptor in a specific tissue or cell that was achieved by the effective dose. The maintenance amount can be administered in the form of a single dose, or a series of doses separated by intervals of days or weeks (divided doses).

Second or subsequent administrations can be administered at a dosage which is the same, less than or greater than the initial or previous dose administered to the subject. A second or subsequent administration is preferably during or immediately prior to relapse or a flare-up of the condition. For example, the second and subsequent administrations can be given between about one day to 30 weeks from the previous administration. Two, three, four or more total administrations can be delivered to the subject, as needed.

Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The present invention will now be illustrated by the following examples, which are not to be considered limiting in any way.

### EXAMPLES

### Example 1

The expression of ghrelin and its receptor was characterized in a model of colonic inflammation, i.e., TNBS-induced colitis. This experiment examined whether ghrelin and its receptor are involved in intestinal inflammation.

Animals

CD1 mice were used in the experiments. Animals were transported in a restraining cage. All experiments were carried out in accordance with NIH standards of care and use of laboratory animals.

Euthanasia was performed by sodium pentobarbital, a commonly used method of anesthesia and euthanasia consistent with the recommendation of the AVA Panel of Euthanasia.

TNBS Colitis

The procedure for TNBS colitis was performed as previously described (Mazelin, L. et al., Life Sci., 63:293-304 (1998)). Briefly, after an overnight fasting, all animals were lightly anaesthetized by an intraperitoneal injection of pentobarbital sodium (70 mg/kg). A polyethylene cannula (Intramedic PE-20 tubing, Becton Dickinson, Parsippany, N.J.) was introduced into the colon (˜3.0 cm) via the anus.

A solution (100 μl) of 40% ethanol (vehicle) or ethanol-containing TNBS (100 mg/kg; Fluka, Ronkonkoma, N.Y.) was instilled into the colon using a 1 ml syringe. Animals were held head down for one minute after the enema to ensure the permanence of the TNBS solution into the colon. Mice were then returned to their cages and received standard pelleted chow and tap water ad libitum. Mice were sacrificed at specific days as outlined below. After recording the body weight, the whole colon was removed, opened longitudinally, washed in ice-cold saline and its length (cm) and weight (mg) recorded.

Colons were examined under a dissecting microscope by two blinded independent investigators for evaluation of the macroscopic damage score (scale 0-10) using previously described parameters (Mazelin, L. et al., Life Sci., 63:293-304 (1998)). Scoring was as follows: 0, no damage; 1, hyperemia without ulcers; 2, hyperemia and thickening of the bowel wall without ulcers; 3, one ulcer without thickening of the bowel wall; 4, sites of ulceration or inflammation; 5, more than two sites of inflammation or one site extending over 0.5 cm; 6-10, damage extending at least 1 cm with the score increasing by 1 for each additional 0.5 cm of involvement.

The colon was also fixed in 4% paraformaldehyde for histologic examination. Longitudinal sections (10 μm thick) were cut and stained with hematoxylin and eosin and histologic evaluation was performed in colonic sections 3 cm above the anus as previously described (Neurath, M. F. et al., J. Exp. Med., 182:1281-1290 (1995)). Blood corticosterone and ghrelin levels were measured in blood taken by retro-orbital eye bleeding before the induction of inflammation (basal levels) and following TNBS-induced colitis every day for the duration of the experiment.

RNA Extraction and Real Time PCR

Total RNA were prepared using a standard procedure described previously (Chomczynski, P. and Sacchi, N., Anal. Biochem., 162:156-159 (1987)). Equal amount of total RNA was used to detect the expression of ghrelin and GHS-R mRNA by regular RT-PCR, and to quantify the levels of ghrelin and GHS-R mRNA by real time PCR.

Ghrelin and GHS-R mRNA were detected as previously described (Date, Y. et al., Endocrinology, 141:4255-4261 (2000)).

The procedure for real time PCR was according to manufacture's instructions using single step real time PCR reagent (Applied Biosystem Co.). The primers for mouse GHS-R were: 5′-CGTCCGCCTCTGGCAGTA-3′ (forward) (SEQ ID NO: 1), 5′-TGGAA GAGTTTGCAGAGCAGG-3′ (reverse) (SEQ ID NO:2) and 5′-/TET/CGGCCCTGGAACTTCGGCG/36-TAMTph/-3′ (probe) (SEQ ID NO:3). The primers for mouse ghrelin were: 5′-AGCCCAGCAGAGAAAGGAATC-3′ (forward) (SEQ ID NO:4), 5′-AGCCAGCCTTCCAGAGCTC-3′ (reverse) (SEQ ID NO:5) and 5′-/5TET/AAGAAGCCACCAGCTAAACTGCAGCCA/36-TAMTph/-3′ (probe) (SEQ ID NO:6).

Expression of Ghrelin and its Receptor in Normal Intestinal Tissue

To examine whether ghrelin and its receptor are expressed in the intestine of normal animals, total RNA was isolated from the small intestine of Wistar rats and expression of ghrelin and its receptor was determined by RT-PCR as described above. The data show that both ghrelin and the receptor (GHS-R) are expressed in normal small intestinal tissue. The sequences of these PCR fragments were confirmed by DNA sequencing analysis. These results confirm previous findings for presence of ghrelin and its receptor in normal intestine (Date, Y. et al., Endocrinology, 141:4255-4261 (2000); Hosoda, H. et al., Biochem. Biophys. Res. Commun., 279:909-913 (2000); Wang, G. et al., Regul. Pept., 105:75-81 (2002); Sakata, I. et al., Peptides, 23:531-536 (2002)).

Expression of Ghrelin and Its Receptor in TNBS-Induced Colitis

To study whether activation of the receptor for ghrelin is involved in the development of intestinal inflammation, a TNBS model of colitis was utilized. The studies were conducted in CD1 mice that were rectally infused with TNBS (100 mg/kg), vehicle control (40% ethanol) (˜3.0 cm from the anal verge) or untreated (without treatment) (normal), as previously described (Castagliuolo, I. et al., Br. J. Pharmacol., 136:271-279 (2002)). After recording body weight of individual animals, the colon was removed and processed for macroscopic score analysis.

All TNBS-treated animals showed typical weight loss, diarrhea and severe ulceration. In contrast, the buffer-injected mice gained similar weight to the normal mice and showed no detectable macroscopic damage.

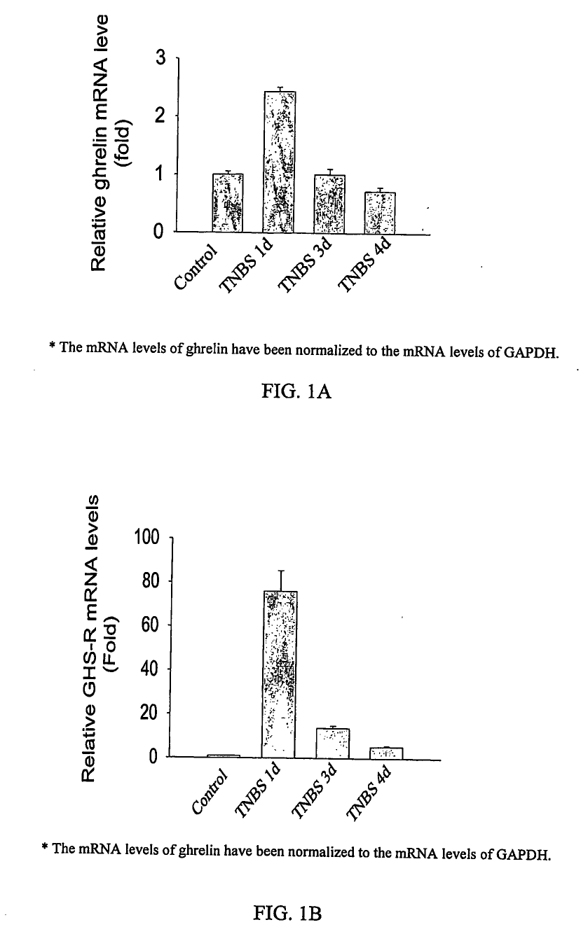

To examine the expression of ghrelin and its receptor (GHS-R), total RNA was isolated from the removed colons and their mRNA levels were determined by a quantitative real time PCR technique. The results showed that the levels of ghrelin mRNA increased 2.4 fold one day after TNBS infusion as compared to the control (FIG. 1A). Surprisingly, the levels of GHS-R increased up to 76 fold one day after TNBS treatment and remained increased 5-6 fold 4 days after TNBS treatment as compared to control (FIG. 1B). This significant upregulation of expression of ghrelin and its receptor (GHS-R), combined with the results of experiments described below, suggest that ghrelin and its receptor play a role in TNBS-induced colonic inflammation.

### Example 2

### Inhibition of Ghrelin-Induced MAP Kinase Phosphorylation in a Dose-Dependent Manner in Vitro by Anti-Ghrelin IgG or Ghrelin Receptor Antagonist

To examine whether an anti-ghrelin IgG or a GHS-R antagonist [D-lys-3]-GHRP-6 blocks ghrelin-mediated responses in experimental colitis, experiments were first performed to test the efficiency of these reagents using in vitro assays. Since ghrelin was previously shown to activate MAP kinase in human HepG2 cells, the effect of the anti-ghrelin antibody or [D-lys-3]-GHRP-6 on ghrelin-induced MAP kinase activation was determined. For these experiments, rat full-length GHS-R cDNA was cloned from rat brain RNA and then subcloned into the retroviral expression vector pMMP. The retroviruses expressing rat GHS-R were used to infect normal rat intestinal epithelial cell line IEC-6 cells (from ATCC). The resulting cell line was named IEC-GHS-R cells.

The response of the IEC-GHS-R to rat ghrelin was determined using this MAP kinase assay. Briefly, serum starved IEC-GHS-R cells were treated with ghrelin (10⁻⁷ M) for various times. Equal amounts of cell extracts were subjected to western blot analysis using the phospho-MAPK specific antibody. The data show that ghrelin induces MAP kinase phosphorylation in a time-dependent manner.

To determine the effect of an anti-ghrelin IgG or a GHS-R antagonist [D-lys-3]-GHRP on ghrelin-induced MAPK phosphorylation, IEC-GHS-R cells were pretreated with the anti-ghrelin IgG (2 and 5 μg/ml) or with normal rabbit IgG or [D-lys-3]-GHRP-6 (1-20 nM) for 10 minutes and then treated with rat ghrelin for 5 minutes. MAP kinase phosphorylation was then determined. The results indicate that both the anti-ghrelin IgG and the GHS-R antagonist [D-lys-3]-GHRP inhibited ghrelin-induced MAPK phosphorylation in a dose-dependent manner. These results show that an anti-ghrelin IgG or a ghrelin receptor antagonist [D-lys-3]-GHRP-6 can efficiently inhibit ghrelin responses in intestinal epithelial cells. In particular, the results show that both an anti-ghrelin IgG and the ghrelin receptor antagonist [D-lys-3]-GHRP-6 can inhibit ghrelin-induced MAP kinase phosphorylation in a dose-dependent manner in vitro.

### Example 3

The molecular mechanism by which ghrelin stimulates IL-8 gene expression in colonocytes was studied. Additionally, the role of NF-κB in this effect was determined.

IL-8 Measurements

IL-8 protein levels in colonic epithelial cell conditioned media were determined by a double-ligand enzyme-linked immunosorbent assay (ELISA) using goat anti-human IL-8 (R & D Systems Inc., Minnesota) as described previously (Linevsky, J. K. et al., Am. J. Physiol., 273:G1333-G1340 (1997)).

IL-8 Promoter-Luciferase Assay

Construction of a reporter construct containing 1521 bp (nucleotides −1481 to +40) of the promoter region of human IL-8 gene has been described previously (Warny, M. et al., J. Clin. Invest., 105:1147-1156 (2000)). IL-8 reporter constructs containing mutations in NF-κB, AP-1 or C/EBP sites have been described previously (Zhao, D. et al., J. Biol. Chem., 276:44464-44471 (2001)). To determine the IL-8 promoter activity in response to ghrelin, NCM-GHSR cells were seeded in 12-well plates (0.2×10⁶ cells/well) overnight and transiently transfected using Effectene Transfection Reagent (Qiagen) with IL-8 promoter luciferase constructs or a control luciferase construct pRL-TK (Promega) or other DNA constructs as indicated. Transfected cells were serum starved for 24 hours followed by exposure to ghrelin for various times. Firefly and Renilla luciferase activities in cell extracts were measured using Dual-Luciferase Reporter Assay System (Promega). The relative luciferase activity were then calculated by normalizing IL-8 promoter-driven Firefly luciferase activity to control Renilla luciferase activity. Data from all experiments are presented as the relative luciferase activity (mean±SE).

Activation of Ghrelin Receptor (GHS-R) Stimulates Expression of Interleukin-8 (IL-8)

The preliminary results that ghrelin receptor is significantly upregulated in TNBS-induced colitis suggest that the receptor plays an important role in intestinal inflammation. To determine whether ghrelin has any proinflammatory effects, the effect of activation of the receptor on expression of inflammatory cytokines was examined. IL-8 is a potent chemotactic factor for neutrophils and critical for formation of intestinal inflammation. Accordingly, IL-8 expression was used as a first readout for potential ghrelin-mediated inflammatory responses. First, GHS-R cDNA was cloned from human brain. The identity of the cDNA was confirmed by sequencing analysis. The cDNA was then cloned into a retroviral expression vector containing an epitope HA. The resulting plasmid was named pMMP-HA-GHSR. The expression of human GHS-R was also confirmed by western blot analysis using anti-HA IgG.

Next, it was examined whether ghrelin receptor is endogenously expressed in human colonic epithelial cell lines, including a non-transformed human colonic epithelial cell line NCM460 and transformed human colonic epithelial cell lines HT29 and Caco-2 cells. A fragment of the same size as in human brain was detected in the human colonic epithelial cells, including NCM460, HT29 and Caco-2. The results show that ghrelin receptor is expressed in human colonic epithelial cells but at a very low level, consistent with the low levels of GHS-R levels in normal mouse colon (FIG. 1B). These results indicate that stimulation with ghrelin does not mediate IL-8 gene expression in these cell lines.

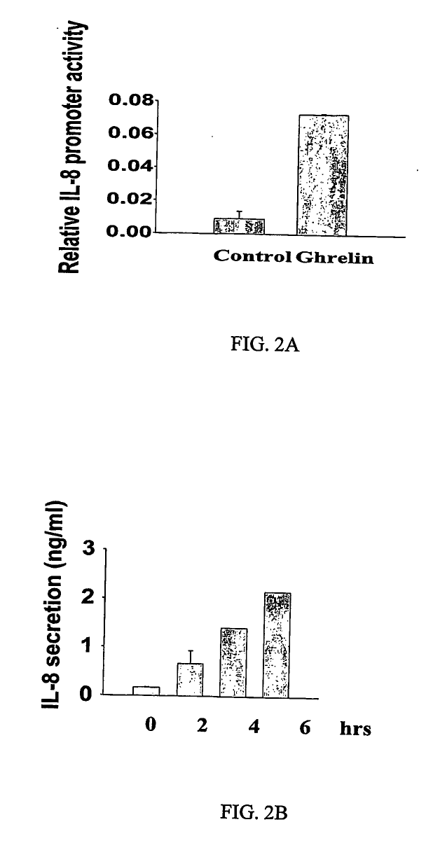

To determine whether increased ghrelin receptor levels are required for IL-8 expression in response to ghrelin, this ghrelin receptor was overexpressed in NCM460 cells using a retroviral expression vector since ghrelin receptor was dramatically upregulated in TNBS colitis (FIG. 1B) (i.e., endogenous levels of GHS-R were 76-fold increased 24 hours after induction of TNBS colitis in NCM460 cells transfected with a GHS-R-expressing plasmid (NCM-GHSR cells)). NCM460 cells were transiently transfected with pMMP-HA-GHSR or a control plasmid pMMP-LacZ along with IL-8 promoter-luciferase construct plus an internal control construct. The transfected cells were stimulated with ghrelin for 6 hours and the IL-8 promoter activity was measured. The results indicate that ghrelin significantly stimulates IL-8 promoter activity (approximately 8 fold) (FIG. 2A).

To determine whether ghrelin stimulates IL-8 protein production, retroviruses expressing human GHS-R were prepared using a previously described procedure (Zhao, D. et al., J. Biol. Chem., 276:44464-44471 (2001)). NCM460 cells were infected with the GHS-R-expressing viruses. The resulting cells were named NCM-GHS-R cells. NCM-GHS-R cells were serum starved and then treated with ghrelin (10⁻⁷ M) for various times. IL-8 secretion in the conditioned media was measured. The data show that ghrelin significantly stimulates IL-8 secretion in NCM-GHS-R cells (FIG. 2B). The results show that stimulation of GHS-R in NCM-GHS-R cells with ghrelin increases IL-8 gene expression as determined by luciferase reporter assay and IL-8 secretion (FIGS. 2A and 2B). These results indicate that the proinflammatory effect of ghrelin requires high levels of expression of its receptor. This is consistent with the recent observation for the requirement of high levels of substance P and neurotensin receptor expression for increased IL-8 gene and protein expression to occur following ligand exposure (Zhao, D. et al., J. Biol. Chem., 276:44464-44471 (2001)).

Involvement of the NF-κB Pathway in Ghrelin-Induced IL-8 Gene Expression

NF-κB is a transcription factor that is ubiquitously expressed and regulates the expression of numerous genes involved in the immune system and the inflammatory response (Silverman, N. and Maniatis, T., Genes Dev., 15:2321-2342 (2001); Ghosh, S. et al., Annu. Rev. Immunol., 16:225-260 (1998); Schmid, R. M. et al., Gut, 43:587-588 (1998); Ellis, R. D. et al., Inflamm. Res., 47:440-445 (1998); Ardite, E. et al., Br. J. Pharmacol., 124:431-433 (1998)). It has been demonstrated that the NF-κB/IκB pathway is critical for the expression of proinflammatory effects of several neuropeptides, such as neurotensin (Zhao, D. et al., J Biol. Chem., 276:44464-44471 (2001)), and substance P (Lieb, K. et al., J. Immunol., 159:4952-4958 (1997); Zhao, D. et al., Biochem. J, 368:665-672 (2002)).

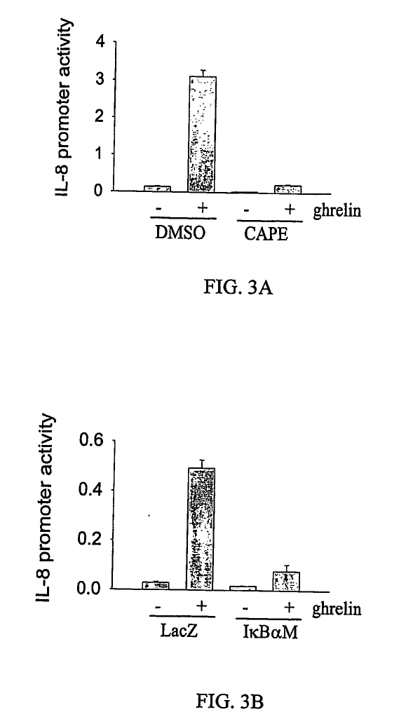

To examine whether ghrelin-induced IL-8 gene expression involves the NF-κB pathway, NCM-GHS-R cells were transiently transfected with IL-8-luciferase construct along with an internal control plasmid. The quiescent cells were pretreated with 10 μg/ml CAPE (a pharmacological inhibitor of the NF-κB pathway) for 30 minutes and treated with ghrelin (10⁻⁷M) for 4 hours. IL-8 promoter activity was measured. The data show that pretreatment with CAPE inhibited ghrelin-induced IL-8 promoter activity (FIG. 3A).

To confirm the involvement of the NF-κB pathway in the response, a molecular approach was used by employing an endogenous inhibitor of NF-κB, IκBα NCM-GHS-R cells were transiently transfected with an IL-8-luciferase construct along with an internal control plasmid, as well as a super-repressor IκBαM- or LacZ (control)-expressing plasmid. The transfected cells were serum starved and then treated with ghrelin (10⁻⁷ M) for 4 hours. IL-8 promoter activity in cell extracts was then measured. The results indicate that expression of IκBα significantly inhibited ghrelin-induced IL-8 promoter activity (FIG. 3B).

### Example 4

### Ghrelin Receptor Antagonism Inhibits C. difficile Toxin A-Induced Fluid Secretion

Clostridium difficile, via release of two toxins, toxin A and toxin B, is the causative agent of antibiotic associated colitis and inflammatory diarrhea in animals and humans (Kelly, C. P. et al., N. Engl. J. Med., 330:257-262 (1994)). It has been shown that C. difficile toxin A induces intestinal fluid secretion and inflammation by activating neurons and immune cells of the intestinal lamina propria (Pothoulakis, C. and Lamont, J. T., Am. J. Physiol. Gastrointest. Liver Physiol., 280:G178-G183 (2001)).

To examine whether ghrelin and its receptor play a role in the pathophysiology of toxin A-induced intestinal fluid secretion in vivo, mice weighing 25-30 grams were housed under controlled conditions on a 12-12 hour light dark circle. Mice were fasted (16 hours) and then pretreated by intraperitoneal injection of 0.2 ml phosphate buffer saline (PBS) containing 70 μg of the ghrelin receptor antagonist [D-lys-3]-GHRP-6 or PBS alone (vehicle). After 30 minutes animals were anesthetized with a mixture of ketamine (0.9 ml) and xylazine (0.1 ml) in 9 ml of sterile water at a dose of 0.15 ml/20 gm body weight. A laparotomy was then performed and a 3-5 cm-long loop was formed at the terminal ileum as previously described (Pothoulakis, C. and Lamont, J. T., Am. J. Physiol. Gastrointest. Liver Physiol., 280:G178-G183 (2001)). Loops were then injected with either 0.1 ml of 50 mM Tris-Cl (pH 7.4) containing 10 μg of purified toxin A or buffer alone (control). The abdomen was then closed and animals were placed on a heating pad at 37° C. for the duration of the experiment. After 4 hours, animals were sacrificed with CO₂ inhalation and fluid secretion was estimated as the loop weight to length ratio, as previously described (Pothoulakis, C. and Lamont, J. T., Am. J. Physiol. Gastrointest. Liver Physiol., 280:G178-G183 (2001); Pothoulakis, C. et al., Proc. Natl. Acad. Sci. USA, 91:947-951 (1994); Castagliuolo, I. et al., J. Clin. Invest., 103:843-849 (1999)).

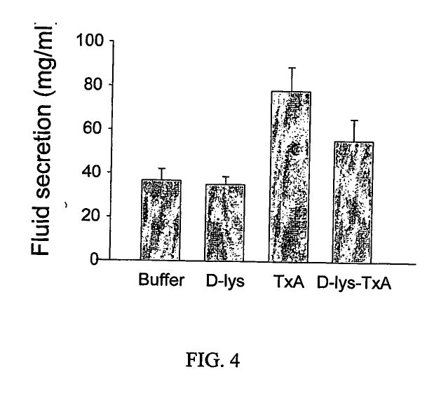

The results show that luminal injection of toxin A in vehicle-treated animals stimulated increased fluid secretion compared to buffer injection (FIG. 4). Administration of D-lys-GBRP-6 had no significant effect in basal fluid secretion in buffer-exposed loops (FIG. 4). However, D-lys-GHRP-6 inhibited toxin A-induced fluid secretion by approximately 50%, suggesting that ghrelin may participate in the pathophysiology of toxin A-mediated intestinal secretion (FIG. 4). Since previous results indicated that C. difficile toxin A-induced intestinal fluid secretion is mediated by proinflammatory neuropeptides and inflammatory mediators released in the intestine during toxin A-mediated enteritis (Pothoulakis, C. and Lamont, J. T., Am. J. Physiol. Gastrointest. Liver Physiol., 280:G178-G183 (2001)), the results herein also suggest that ghrelin is involved in the athophysiology of intestinal inflammation.

### Example 5

### Upregulation of Functional GHS-R-1a mRNA in Colons of Human Patients with Crohn's Disease (CD) or Ulcerative Colitis (UC)

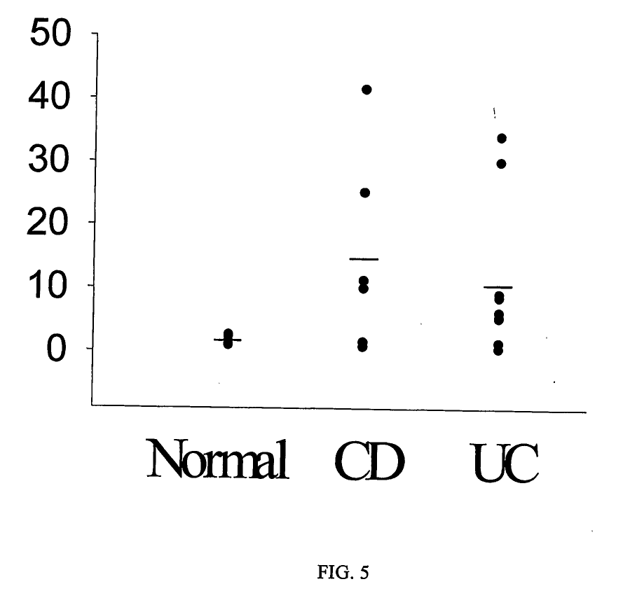

To examine whether upregulation of ghrelin and GHS-R in TNBS colitis is relevant to the pathophysiology of human IBD, a determination was made of whether expression of GHS-R is altered in the colons of patients with CD or UC as compared to noninflamed colons. Total RNA was isolated from the inflamed and non-inflamed colons of patients (6 control, 6 CD and 9 UC). Equal amounts of RNA were used to determine the levels of GHS-R-1a mRNA by quantitative real time PCR using human GHS-R-1a specific primers. The data show that the levels of GHS-R-1a mRNA are significantly higher in the colons of patients with either CD (8.9 fold, p<0.05) or UC (6.7 fold, p<0.05) as compared to the control (FIG. 5).

### Example 6

### NCM460-GHS-R Cell Line

The coding region of GHS-R-1a was isolated from human brain mRNA (Strategene, CA) by RT-PCR using the primers 5′-GCCTCTCACCTCCCTCTCTTTC-3′ (forward) (SEQ ID NO: 9) and 5′-CTCGCAATGTGCTAGGTCATG-3′ (reverse) (SEQ ID NO: 10). The PCR fragment was subcloned into a TA cloning vector (Invitrogen) and its identity was confirmed by DNA sequencing. The coding region was then isolated from the TA vector and subcloned into the pMMP retroviral vector (kindly provided by Dr. Richard A. Mulligan, Children's Hospital, Boston). Preparation of retroviruses expressing GHS-R and infection of human colonic epithelial cells NCM460 were done according to previously described procedure (Zhao D et al., J Biol. Chem., 276(48):44464-44471 (2001)). This cell line is an example of a cell line that can be used in the methods disclosed herein for identifying or screening for a ghrelin antagonist and/or a ghrelin receptor antagonist.

### Example 7

Additional experiments can be performed to further examine the involvement of ghrelin and its receptor in intestinal inflammation. In addition to TNBS-induced colitis, the expression of ghrelin and its receptor can also be characterized in two additional models of colonic inflammation (DSS-induced colitis and C. difficle toxin A-induced enteritis). The following describes these additional experiments.

TNBS Colitis

The procedure for TNBS colitis is as described above.

DSS Colitis

Mice receive standard pelleted chow and tap water ad libitum for the duration of the experiments. Colitis is induced by DSS 5% molecular weight 40,000 (ICN Biochemicals) in drinking water. Control mice receive normal drinking water. After various days, colon and other portions of intestine are removed and opened longitudinally. Macroscopic and histological damage score is determined using a previously described scoring system for DSS colitis (Cooper, H. S. et al., Lab. Invest., 69:238-249 (1993)).

Toxin A Enteritis

The procedure for this model of entero-colitisis is followed as previously described Castagliuolo, I. et al., J. Clin. Invest., 101:1547-1550 (1998)). Briefly, overnight fasted mice are anesthetized and a laparotomy is performed. A 5-c, long closed distal ileal loop or colonic loop is formed and injected with either buffer control (50 mM Tris, pH7.4) or 5-10 μg of toxin. Animals are then killed at different times up to 4 hours. The ileal loop is removed, their weights and lengths are recorded, and fluid secretion is assessed by the ratio of loop weight (mg) to length (cm). Part of the loop is fixed and processed for in situ hybridization and immunohistochemistry analysis. The remaining loop is used to isolated total RNA for quantification of mRNA levels by real time PCR.

Ghrelin RIA Assay

Tissue and plasma for ghrelin assay are processed using a procedure described by Lee et al. (Lee, H. M. et al., Endocrinology, 143:185-190 (2002)) and ghrelin levels in extracts are then determined by radio-immuno assay (RIA) using commercially available kits (Pheonix Pharmaceuticals, Inc.).

Measurement of ACTH and Corticosterone

Plasma ACTH and corticosterone are determined as previously described (Castagliuolo, I. et al., Am. J. Physiol. Gastrointest. Liver Physiol., 280:G539-G545 (2001)) using commercially available kits (IncStar and ICN, respectively).

RNA Extraction and Real Time PCR

RNA extraction and real time PCR are as described in Example 1.

In Situ Hybridization for Ghrelin and GHS-R mRNA

Total RNA used for amplification of 400-500 bp human cDNA probes for ghrelin and GHS-R were purchased from Strategene Co. Mouse brain RNA was directly isolated from mouse whole brain. All primers for amplifying cDNAs probes for human and mouse ghrelin and GHS-R were based on published sequences (Date, Y. et al., Endocrinology, 141:4255-4261 (2000); Papotti, M. et al., J. Clin. Endocrinol. Metab., 86:5052-5059 (2001)).

The PCR probes were subcloned into Topo TA cloning vector (Invitrogen) and confirmed by DNA sequencing analysis. The plasmids are used to make sense and antisense riboprobes using digoxigenin (DIG) according to the manufacture's instructions (Boehringer Mannheim, Indianapolis, Ind.). In situ hybridization is performed as previously described (Castagliuolo, I. et al., J. Clin. Invest., 103:843-849 (1999)). Briefly, tissue sections are fixed in 4% paraformaldehyde-PBS for 5 minutes and acetylated for 15 minutes at room temperature in 0.25% acetic anhydride. Sections are then incubated with DIG-labeled probes in a moisture chamber at 53° C. overnight in the presence of hybridization buffer (50% formamide, 10% dextran sulfate, 4×SSC, 1× Denhart's solution, 0.1 mg/ml salmon sperm DNA, 0.125 mg/ml tRNA, 0.1 mg/ml DTT). After hybridization, slides are digested with RNase (20 μg/ml) for 1 hour at 37° C. and washed with 2×SSC, 1×SSC and 0.1×SSC for 1 hour each at 50-58° C. Slides are then incubated with a fluorescein labeled sheep anti-DIG conjugate (Boehringer Mannheim) at a dilution of 1:6 in blocking serum (1% donkey serum, 2% BSA, 0.05 M NH₄Cl, 0.1% Tween-20). After multiple washing in 1×TBS (50 mM Tris, 0.15 M NaCl, pH7.5), sections are mounted and the images are viewed by a confocal microscope. Sense riboprobes are used as controls.

Identification of Cell Types By Immunohistochemistry

The type of cells expressing ghrelin and its receptor are identified using double labeling techniques combining in situ hybridization and immunohistochemistry. In situ hybridization is used to identify ghrelin and its receptor mRNA positive cells as described above. Immunohistochemistry is performed to identify the various cell types containing ghrelin or its receptor. Staining for cytokeratin (an epithelial structural protein) is performed by a rabbit anti-cytokeratin antibody (Accurate Chemical and Sciences). Monoclonal mouse anti-CD4 and anti-CD8 is used to identify T-cells, monoclonal mouse anti-CD10 is to identify B-lymphocytes and monoclonal mouse anti-CD14 is used to identify macrophages (all antibodies available from Serotec). Neurons are identified by an antibody directed against the neurofilament specific protein of 200 kDa (Accurate Chemical and Sciences). For secondary antibodies, IgG labeled with rhodamine and fluorescein (Jackson Labs) are used.

Neutrophils and eosinophils are identified by Wright staining. Immunohistochemistry is performed as previously described (Castagliuolo, I. et al., J. Clin. Invest., 103:843-849 (1999)). Briefly, sections are washed in 1×TBS and then fixed in 4% paraformaldehyde in PBS (pH8.5). Sections are next incubated for 30 minutes in 1% hydrogen peroxide and then for 30 minutes in blocking serum (1% donkey serum, 2% BSA, 0.05 M NH₄Cl, 0.1% Tween-20). The slides are then incubated with rabbit polyclonal antibodies against ghrelin (Pheonix Pharmaceuticals) or GHS-R (Alpha Diagonostic International, Inc.) or normal rabbit IgG for 1 hour. After washing three times (10 minutes each) in 1×TBS, sections are incubated for 1 hour with FITC-conjugated anti-rabbit IgG. After washing in TBS, the slides are mounted and photographed using confocal microscope.

To identify the cell type that express ghrelin or its receptor, the above FITC-labeled slides are further incubated with a cell type specific antibody followed by incubation with a rhodomine-conjugated anti-mouse or rabbit IgG. Sections are then mounted and photographed using a confocal microscope.

Laser Capture Microdissection

To quantify ghrelin and GHS-R mRNA levels in different cell populations, cells expressing ghrelin or its receptor (identified from immunohistochemistry and in situ hybridization as described above), are isolated by laser capture microdissection. Tissue specimens are mounted in OCT embedding compound (Miles), frozen using liquid nitrogen and stored at −80° C. until use. The tissues are then sectioned (8 μm) and mounted onto SuperFrost Plus glass slides (Fisher Scientific) at room temperature. Sections are then immediately fixed in 70% ethanol for 45 seconds at room temperature, washed in 50% ethanol and stained using 0.1% methylene blue in 10% ethanol for 15 seconds at room temperature. After staining, the sections are incubated successively in water, 70% ethanol, 95% ethanol, 100% ethanol and xylene. The particular type of cells expressing ghrelin or its receptor as shown by immunohistochemistry and in situ hybridization are then isolated from each tissue section using a PixCell II Laser Capture Microdissection System (Arcturus, Mountain View, Calif.). For each experiment, approximately 600-1000 cells per tissue sample are captured using CapSure HS LCM Caps (Arcturus). Total RNA from each sample are then prepared using a PicoPure RNA isolation kit (Arcturus) followed by treatment with RNase-free DNase I (Sigma) to eliminate genomic DNA contamination. Equal amount of total RNA is used to determine the levels of ghrelin and its receptor mRNA by quantitative real time PCR.

Expression of Ghrelin and its Receptor in TNBS-Induced Colitis

Mice (CD1, C57BL/6 and/or BALB/c strains) are used. Briefly, mice are anesthetized and intracolonically infused with 100-250 mg/kg of TNBS to induce colitis. Animals are euthanized 1, 2, 3 and 4 days after treatment or for acute colitis or treated with a second TNBS enema 7 day later (100 mg/kg) and then sacrificed after an additional 5, 15 and 20 days. Controls include saline-treated and ethanol (vehicle)-treated animals. The weight of all animals are monitored before and after infusion of TNBS daily. Blood is collected for ghrelin and corticoid measurements every day. Levels of circulating and locally expressed cytokines (IL-6, TNFα and IL-1β), body weight change, colonic secretion and degree of colonic inflammation (macroscopic damage score, quantitative histopathology and MPO activity) are also measured.

For ghrelin and ghrelin receptor expression, colon are washed in saline. Total RNA is isolated for quantification of mRNA levels of ghrelin and its receptor, GHS-R. The protein levels of ghrelin are also measured in the colon by radioimmuno assay (RIA) using commonly available reagents. Colon is also processed for in situ hybridization and immunohistochemistry analyses to localize expression of ghrelin and GHS-R. Details for these measurements are described above.

Expression of Ghrelin and its Receptor in DSS Colitis

In contrast to TNBS colitis, acute DSS colitis does not require the presence of T cells to induce intestinal damage (Dieleman, L. A. et al., Gastroenterology, 107:1643-1652 (1994)). The DSS model, a commonly used animal model to mechanisms of colitis, is used to determine the expression of ghrelin and GHS-R genes. Briefly, mice of either sex are subjected to 5 days DSS treatment. Animals are euthanized every day for the 5 day period and blood is collected for serum corticosteroid and ghrelin measurements. Levels of circulating cytokines (IL-6, TNFα, IL-1β) and degree of colonic inflammation are also measured. For ghrelin and GHS-R measurements, different portions of intestine are removed and opened longitudinally. Macroscopic damage is immediately assessed. Colonic tissues are processed to quantitatively determine the levels of ghrelin and GHS-R mRNA using quantitative real time PCR, their protein levels using RIA; and to localize the expression of ghrelin and GHS-R using both in situ hybridization and immunohistochemistry.

Expression of Ghrelin and Its Receptor in C. difficile Toxin A-Induced Enteritis

The toxin A ileal and colonic loop model has been used successfully to define the role of neuro-immune interactions in the development and progress of intestinal inflammation (Castagliuolo, I. et al., J. Clin. Invest., 103:843-849 (1999); Castagliuolo, I. et al., J. Clin. Invest., 101:1547-1550 (1998); Wlk, M. et al., Gastroenterology, 123:505-515 (2002); Pothoulakis, C., Ann. N.Y. Acad. Sci., 915:347-356 (2000); Pothoulakis, H. et al., Compr. Ther., 11:68-73 (1985)). Thus, this model can be used to determine expression of ghrelin and its receptor, GHS-R, which can then be correlated with the degree of secretion and inflammation. Briefly, mice are anesthetized and one 5 cm loop is constructed either in the ileum or colon and injected either with purified toxin A (5 μg) or with buffer (50 mM Tris-Cl, pH7.4) alone. After 15, 30, 60, 120 and 240 minutes, animals are sacrificed and loops are removed to isolate total RNA for determination of ghrelin and GHS-R mRNA levels by real time PCR. Loops are also processed for localization of expression of ghrelin and GHS-R by in situ hybridization and immunohistochemistry. Serum levels of ghrelin and corticosteroid are determined at the indicated time intervals. Fluid secretion, and the degree of inflammation, measured by quantitative histopathology and changes in neutrophil myeloperoxidase (MPO) levels, are determined. Colonic mucosal scrapings are also obtained at the same time intervals for evaluation of TNFα and ILβ.

Quantification of Expression of Ghrelin and its Receptor in Specific Cell Types in Colitis Induced by TNBS, DSS and Toxin A

After identifying the cell types that have increased expression of ghrelin and its receptor during formation of colitis by in situ hybridization and/or immuno-histochemistry, these cell types are isolated in ethanol-fixed tissue sections by Laser Capture Microdissection after nuclear staining with methylene blue. Total cellular NA is purified from isolated cells and used to quantify the levels of ghrelin and GHS-R mRNA by real time PCR.

Anticipated Results

Since results from the TNBS colitis model show that expression of both ghrelin and GHS-R mRNA are significantly increased at least in the acute phase of colitis, it is reasonable to expect that this receptor and ghrelin will also be upregulated in the three different colitis models. It is also reasonable to expect that circulating ghrelin will also be increased. Although the expression of GHS-R or ghrelin may differ in different models of colitis because these models involve different pathophysiology, results from these experiments, together with results from the experiments described below, provide valuable information as to whether this receptor participates in the development of a particular colitis.

Fasting increases ghrelin levels. Since overnight fasting is required for the induction of TNBS- and toxin A-induced colitis, increased circulating ghrelin levels may be present, independently of the induction of colitis. Accordingly, ghrelin levels in overnight fasted, but not toxin A- and TNBS-treated animals can be measured, and their ghrelin levels compared to fasted, toxin A- and TNBS-treated mice. Overnight fasting is not part of the protocol for induction of DSS colitis.

Differences in the levels of expression of ghrelin and its receptor between the acute phase (1-3 days) and the chronic phase (12-20 days) of TNBS-induced colitis can be determined. Such differences are expected in light of the preliminary results described herein suggesting an acute rise of ghrelin and ghrelin receptor mRNA expression 1 day after induction of TNBS colitis and a decrease from these increased levels after day 1. To further study the potential mechanism of this response, measurements of corticosteroids can be determined. Since ghrelin has been associated with increased activity of EPA axis via central stimulation of CRH expression (Asakawa, A. et al., Neuroendocrinology, 74:143-147 (2001)), and intestinal inflammation is known to be associated with increased circulating corticosteroids levels (Castagliuolo, I. et al., Am. J. Physiol Gastrointest. Liver Physiol., 280:G539-G545 (2001)), increased corticosteroid levels following induction of colitis are expected. Increased corticosteroids levels may also be responsible for the reduced levels of expression of ghrelin and its receptor after the acute phase of colitis.

Similar experiments can be performed in adrenalectomized mice with or without replacement with glucocorticoid and levels of expression of ghrelin and its receptor under these experimental conditions can be compared.

The levels of ghrelin and GHS-R in isolated intestinal cells from both inflamed and control tissues are quantified. Quantitative information on the expression levels of ghrelin and its receptor in a particular cell type provide support for the qualitative results from in situ hybridization and immunohistochemical experiments. Since preliminary evidence indicate that colonic epithelial cells express GHR-R mRNA, particular focus can be given this cell population in the mouse colon.

### Example 8

Additional experiments can also be performed to examine the function of ghrelin and its receptor in the inflammatory process in vivo using an anti-ghrelin IgG and a ghrelin receptor (GHS-R)-specific antagonist. The following describes these additional experiments.

Material and Methods

Animals

As above, CD1, C57BL/6 and BALB/c strains of mice are used in the experiments. Animals are transported in a restraining cage. All experiments are carried out in accordance with NIH standards of care and use of laboratory animals.

In the toxin A protocol, animals are anesthetized by intraperitoneal injection of sodium pentobarbital. The abdomen is then opened and two ileal loops are formed and ligated by silk. Loops are then injected with toxin A or buffer. Animals are kept under mild anesthesia for up to 4 hours depending on the experiment and then sacrificed by decapitation. The only pain encountered by the animals is the pain of the intraperitoneal or intravenous injection through a 27 gauge needle. The TNBS and DSS protocols are described below.

Euthanasia is performed by sodium pentobarbital, a commonly used method of anesthesia and euthanasia consistent with the recommendation of the AVA Panel of Euthanasia.

Effect of the Anti-Ghrelin Antibody and [D-lys-3]-GHRP-6 On TNBS, DSS and Toxin A Colitis A large quantity (milligrams) of the antibody against the C-terminal fragment (13-28, QQRKESKKPPAKLQPR (SEQ ID NO NO:7), human and rat are the same) of ghrelin is prepared commercially from Sigma-Genosys Co. Different doses of the anti-ghrelin IgG or a control pre-immune IgG or [D-lys-3]-GHRP-6 are ip injected to animals and then their effect on colitis is analyzed as described above.

To quantitatively measure inflammation, myeloperoxidase (MPO) activity (a measure of neutrophil infiltration) and levels of the cytokines IL-1β and TNFα are determined in the colonic tissues as described below. To determine the effect of the anti-ghrelin antibody and [D-lys-3]-GHRP-6 on neuropeptide expression, colon is also processed and levels of NT, SP and CRH in extracts are measured as described below.

Myeloperoxidase (MPO) Activity Assay

MPO activity, a measure of neutrophil infiltration is determined according to a modification of the method described by Bradley et al (Bradley, P. P. et al., J. Invest. Dermatol., 78:206-209 (1982)). Briefly tissue samples are homogenized in 50 mM KH₂PO₄ buffer (pH6.0) containing 0.5% hexadecyltrimethyl-ammonium bromide, freeze thawed three times, sonicated for 10 seconds, and centrifuged at 10,000 g for 15 minutes. MPO activity in the supernatant is measured in triplicate by a calorimetric assay in which 30 μl of sample is mixed with 720 μl of 50 mM phosphate buffer containing 0.167 mg/ml O-dianisidine dihydrochloride (Sigma) and 0.0005% hydrogen peroxide (pH6.0). The reaction mixture is incubated for 10 minutes for color development and optical absorbance is read at 460 nM. Results are expressed in MPO units per mg of protein in the supernatant by comparison with a standard curve derived from parallel assay of purified MPO (Calbiochem-Novabiochem Co.).

Cytokine Measurements

To measure the levels of IL-1β and TNFα, tissue sample is homogenized in ice-cold phosphate-buffered saline (pH7.4) containing protease inhibitors. Homogenates are centrifuged at 14,000 g for 10 minutes at 4° C. and the IL-1β and TNFα levels in the supernatants are measured using a commercially available kit (Biosource International, CA) according the manufacturer's instruction. The results are expressed as picograms per milligram of total protein.

Neuropeptide Assays

Neurotensin (Castagliuolo, I. et al., J. Clin. Invest., 103:843-849 (1999)), substance P (Castagliuolo, I. et al., J. Clin. Invest., 101:1547-1550 (1998)) and CRH (Wlk, M. et al., Gastroenterology, 123:505-515 (2002)) in the colon are measured as previously described using the enzymatic immunoassay (EIA) kit (Peninsula Laboratories, CA)

Role of Ghrelin and GHS-R Interaction In Intestinal inflammation

As shown in the results in Example 1 (see FIGS. 1A, 1B, 2A and 2B), ghrelin and GHS-R are upregulated at the early stage of TNBS colitis and activation of GHS-R directly induces IL-8 gene expression in human colonic epithelial cell lines. These results strongly suggest that ghrelin/GHS-R interaction plays an important role in development of intestinal inflammation.

To confirm the role that ghrelin/GHS-R interaction plays in development of intestinal inflammation, a number of approaches can be used to block GHS-R activation and/or expression and then whether intestinal inflammation is inhibited, at least partially, can be examined. These approaches include using (1) an anti-ghrelin IgG, (2) a GHS-R-specific antagonist, and eventually (3) GHS-R-deficient mice.

The first approach is to use an anti-ghrelin antibody. The rabbit polyclonal antibody, directed against the C-terminal fragment (13-28, QQRKESKKPPAKLQPR (SEQ ID NO:7), human and rat are same) of ghrelin (Kojima, M. et al., Nature, 402:656-660 (1999)), was successfully used to block the food intake induced by centrally injected ghrelin (Nakazato, M. et al., Nature, 409:194-198 (2001)). The second approach is to use a GHS-R-specific antagonist [D-lys-3]-GHRP-6 that was shown to efficiently block GHS-R-induced calcium response and food intake (Kojima, M. et al., Nature, 402:656-660 (1999); Nakazato, M. et al., Nature, 409:194-198 (2001)). These first two approaches have been successfully used to inhibit ghrelin-induced MAP kinase phosphorylation in in vitro cell culture experiments as described herein. Thus, it is expected that activation of ghrelin receptor can be blocked in in vivo models with these antagonists.

The third approach involves the creation of GHS-R deficient mice. This third approach provides direct evidence for a proinflammatory role of ghrelin and its receptor in colitis. Such knockout animals can be used in the colitis models described herein.

Expression of neuropeptides such as neurotensin (NT), substance P(SP), corticotropin-releasing hormone (CRH) and their receptors are increased during toxin A enteritis (Castagliuolo, I. et al., J. Clin. Invest., 103:843-849 (1999); Castagliuolo, I. et al., J. Clin. Invest., 101: 1547-1550 (1998); Wlk, M. et al., Gastroenterology, 123:505-515 (2002); Pothoulakis, C. et al., Ann. N.Y. Acad. Sci., 840:635-648 (1998)). In addition, peripheral ghrelin increases central CRH expression (Asakawa, A. et al., Neuroendocrinology, 74:143-147 (2001)). To determine whether ghrelin also regulates expression of CRH, NT and SP in intestine during course of inflammation, a determination is made regarding whether blockade of ghrelin and its receptor interaction is associated with altered levels of NT, SP and CRH in the toxin A colitis model.

Effects of Anti-Ghrelin Antibody and [D-lys-3]-GHRP-6 On TNBS Colitis

TNBS colitis is induced as described above. For the blocking antibody experiment, animals are divided into five groups: control, vehicle (40% ethanol), TNBS alone, TNBS+anti-ghrelin antibody (blocking antibody, 1 mg/kg IP, 30 minutes before and 24 hours after TNBS), and TNBS+control rabbit IgG (1 mg/kg IP, 30 minutes before and 24 hours after TNBS). For the antagonist experiment, animals are divided into three groups: control, vehicle (40% ethanol), TNBS alone, TNBS+[D-lys-3]-GHRP-6 (1 mg/kg IP, 30 minutes before and 24 hours after TNBS). Three days after TNBS administration, body weight is determined, animals are killed, colons are removed and macroscopic damage is scored. Small pieces of tissue are fixed for histologic analysis. The remaining colon is processed for determination of MPO activity and expression of IL-1β and TNFα by ELISA.

Effects of Anti-Ghrelin Antibody and [D-lys-3]-GHRP-6 on Toxin A Enteritis

Toxin A colitis is induced as described above. For the anti-ghrelin antibody experiments, animals are divided into four groups: buffer control (50 mM Tris pH7.4), toxin A alone, toxin A+anti-ghrelin antibody (blocking antibody, 1 mg/kg IP, 30 minutes before toxin A), and toxin A+control rabbit IgG (1 mg/kg IP, 30 minutes before toxin A). For the antagonist experiments, animals are divided into three groups: buffer control (50 mM Tris pH7.4), toxin A alone, toxin A+[D-lys-3]-GHRP-6 (1 mg/kg IP, 30 minutes before toxin A injection). Animals are killed 4 hours after toxin A injection, ileal loops are removed, and fluid secretion is measured. Small pieces of loops are fixed for histologic analysis, and the remaining tissue is then processed for determination of MPO activity and expression of IL-1β and TNFα as well as NT, SP and CRH.

Anticipated Results

As discussed above, results herein show that ghrelin and ghrelin receptor mRNAs are elevated in the acute phase of TNBS-induced colitis in vivo, and ghrelin and ghrelin receptor antagonism inhibits ghrelin-induced MAP kinase activation in vitro. Additionally, as discussed above, results herein also show that ghrelin and ghrelin receptor antagonism inhibited toxin A-induced fluid secretion by approximately 50% (FIG. 4), suggesting that ghrelin may participate in the pathophysiology of toxin A-mediated intestinal secretion. Thus, it is reasonable to expect that blockage of ghrelin-receptor interaction by either anti-ghrelin antibody or GHS-R specific antagonist [)-lys-3]-GHRP-6 will inhibit acute colitis induced by TNBS and toxin A.

Ghrelin and ghrelin receptor antagonism experiments are performed in the acute phase of TNBS-induced colitis (1-3 days) and in the toxin A-induced enterocolitis model (4 hours). If these antagonists show an inhibitory effect, the DSS model and the more chronic phase of TNBS-induced colitis can be examined. If the results demonstrate a protective effect of the ghrelin receptor antagonist, experiments can be performed to determine if these compounds ameliorate established colitis by administering them after the inflammatory stimulus (TNBS or toxin A) is given. Results from these experiments provide information as to whether ghrelin or ghrelin receptor antagonism have therapeutic potential.

If the results indicate that administration of the ghrelin antibody and/or the GHS-R antagonist reduces inflammatory responses in the models of colitis described herein, it is reasonable to conclude that peripheral ghrelin, by interacting with its receptors localized in the intestinal mucosa, possesses these proinflammatory effects. This is because the anti-ghrelin antibody and the peptide antagonist of GHS-R, due to their size and hydrophilicity, should not be able to cross the blood-brain barrier and neutralize ghrelin in the CNS. In this case, experiments can be conducted to determine whether in an in vivo situation ghrelin receptors localized in the colonic mucosa play a proinflammatory role in the development of colitis. In these experiments, 24 hours after induction of colitis with TNBS (at which time, as demonstrated in the experiments above, ghrelin receptor expression is increased), mice are killed and colonic explants are prepared and placed in organ culture. Explants are then exposed to medium containing ghrelin or medium alone and, at different time points, supernatants are collected for measurements of TNFα and IL-1β.

### Example 9

Additional experiments can also be performed to further examine the molecular mechanism by which ghrelin stimulates IL-8 gene expression in colonocytes and the role of NF-κB in this effect. The following describes these additional experiments.

Materials and Methods

IL-8 Measurements

IL-8 protein levels in colonic epithelial cell conditioned media were determined by a double-ligand enzyme-linked immunosorbent assay (ELISA) using goat anti-human IL-8 (R & D Systems Inc., Minnesota) as described previously (Linevsky, J. K. et al., Am. J. Physiol., 273:G1333-G1340 (1997)). Results are expressed as mean±SE (ng/ml).

IL-8 Promoter-Luciferase Assay

To determine the IL-8 promoter activity in response to ghrelin, NCM-GHSR cells are seeded in 12-well plates (0.2×10⁶ cells/well) overnight and transiently transfected using Effectene Transfection Reagent (Qiagen) with IL-8 promoter luciferase constructs or a control luciferase construct pRL-TK (Promega) or other DNA constructs as indicated. Transfected cells are serum starved for 24 hours followed by exposure to ghrelin for various times. Firefly and Renilla luciferase activities in cell extracts are measured using Dual-Luciferase Reporter Assay System (Promega). The relative luciferase activity are then calculated by normalizing IL-8 promoter-driven Firefly luciferase activity to control Renilla luciferase activity. Data from all experiments are presented as the relative luciferase activity (mean±SE).

Electrophoretic Mobility Shift Assays (EMSAs)

Nuclear extracts are prepared for DNA binding assays as described previously (Zhao, D. et al., J. Biol. Chem., 276:44464-44471 (2001)). Cells are washed in PBS, collected into TNE buffer (40 mM Tris (pH 7.4), 1 mM EDTA, 0.15 M NaCl), and centrifuged (5000 g×10 sec). The cell pellets are incubated with buffer A [10 mM Hepes (pH 7.9), 10 mM KCl, 0.1 mM EDTA, 0.1 mM EGTA, 0.5 M PMSF] for 10 minutes before addition of 10% NP40 for an additional 2 minutes. Nuclei are centrifuged (5000 g× for 10 seconds), incubated with buffer B [20 mM Hepes (pH 7.9), 0.4 M NaCl, 1 mM EDTA, 1 mM EGTA, 0.1 mM PMSF] for 45 minutes, and centrifuged at 13000×g for 10 minutes. Nuclear extracts are incubated with poly(dI-dC), band shift buffer [50 mM MgCl₂, 340 mM KCl, and 8 μl of delta buffer (0.1 mM EDTA, 40 mM KCl, 25 mM N-2-hydroxyethylpiperazeine-N′-2-ethananesulfonic acid (HEPES; pH 7.6), 8% Ficoll 400, 1 mM dithiothreitol] at 4° C. for 15 minutes. ³²P-labeled doubled-stranded oligonucleotide probe (100,000 cpm) is then added to the reaction mixture and incubated for 30 minutes on ice. For supershift assays, the appropriate antibody is added to the nuclear extract and incubated at 4° C. for 30 minutes before addition of the probe. Binding of specific nuclear protein to the probe is determined by fractionating the nuclear proteins through a nondenaturing 6% polyacrylamide gel at 200 volts for 2 hours at room temperature in TBE buffer [80 mM Tris-borate, 2 mM EDTA (pH 8.0)]. The gel is dried at 80° C. for 2 hours under vacuum before exposure to X-ray autoradiography film. The NF-κB consensus oligonucleotide (Promega) and the oligonuceotide of IL-8-promoter (−83 bp to −69 bp) containing the κB-like site (−80 bp to −71 bp; GGAATTTCCT (SEQ ID NO:8)) are end-labeled by T4 DNA kinase (New England Biolabs, Beverly, Mass.) and [γ-³²P] ATP (DuPont NEN, Boston, Mass.).

Detection of IκBα Expression and Phosphorylation of p65

Cell extracts are separated by SDS-PAGE and transferred onto nitrocellulose membrane (Millipore) in a buffer containing 25 mM Tris, 192 mM glycine, and 20% methanol for 3 hours at 80 Volts and 4° C. The blots are blocked for 1 hour in TBST (20 mM Tris pH 7.6, 137 mM NaCl, and 0.1% Tween-20) containing 5% dry milk. Blots are then washed in TBST and incubated with antibodies against IκBα (Santa Cruz) or phosphorylated p65 (Cell Signaling, MA) for 1 hour at room temperature with shaking. The blots are washed in TBST and incubated with secondary antibody (Santa Cruz), and proteins visualized with chemiluminescence (Amersham).

Statistical Analysis

Results are expressed as means±SEM. Data are analyzed using the SIGMA-STAT™ professional statistics software program (Jandel Scientific Software, San Rafael, Calif.). Analyses of variance with protected t test (ANOVA) are used for intergroup comparison.

Involvement of the NF-κB Pathway In Ghrelin-Induced IL-8 Gene Expression

To further examine the role of the NF-κB pathway in ghrelin-induced IL-8 promoter activity, dominant negative mutants of IKKα, IKKβ and IκB can be used in the IL-8 promoter-driven luciferase reporter assay and IL-8 secretion assay. Briefly, NCM-GHS-R cells are cotransfected with dominant negative IKKα-, IKKβ- or IκBα- or control LacZ-expressing plasmid along with IL-8-luciferase plasmid and an internal control. Luciferase activity in cell extracts is determined.

To examine the effect of dominant negative IKKα, IKKβ or wild type IκBα on ghrelin-induced IL-8 protein secretion, NCM-GHS-R cells are infected with dominant negative IKKα-, IKKβ- or wild type IκBα-expressing retroviruses followed by serum starvation and stimulation with ghrelin for up to 24 hours. IL-8 secretion in conditioned media is measured.

To further demonstrate that a NF-κB binding site in the IL-8 promoter is required for this response, IL-8 promoter constructs containing site-directed mutation in a NF-κB binding site (Lieb, K. et al., J. Immunol., 159:4952-4958 (1997)) are used. For this experiment, NCM-GHS-R cells are transiently transfected with wild type or the κB-mutant IL-8 promoter-luciferase constructs and quiescent cells are treated with ghrelin for 4 hours. Luciferase activity in cell extract is measured.

Ghrelin Stimulation of the NF-κPathway

As described above, the NF-κB pathway can be regulated at several levels, including degradation of IκB, and nuclear translocation and phosphorylation of the NF-κB subunit p65. Accordingly, experiments are performed to examine whether ghrelin affects each of these NF-κB-related steps.

First, to determine whether ghrelin stimulates nuclear translocation and DNA binding activity of NF-κB, NCM-GHS-R cells and the parental NCM460 cells are serum starved and treated with ghrelin (10⁻⁷ M) for various times to identify the maximal activation time point of ghrelin (10⁻⁹ M to 10⁻⁶M) for the optimal dose. Nuclear extracts are prepared and DNA binding activity of NF-κB is then performed using both a consensus NF-κB probe as well as IL-8-specific NF-κB-responsive element(s) by electrophoretic mobility shift assay (EMSA).

To examine whether ghrelin causes or modulates degradation of IκBα or phosphorylation of p65, NCM-GHS-R cells and NCM460 cells are serum starved and treated with ghrelin (10⁻⁷ M) for various times. Equal amounts of total cell extracts are subjected to western blot analysis using antibodies against IκBα (Santa Cruz, Calif.) and phosphorylated p65 (Cell Signaling, MA).

Identification of Upstream Molecules of NF-κB Activated By Ghrelin

Previous studies have shown that activation of GHS-R in rat somatotrophes results in release of intracellular calcium (Herrington, J. and Hille, B., Endocrinology, 135:1100-1108 (1994)), and increased phospholipase C and protein kinase C (PKC) activities (Cheng, K. et al., Endocrinology, 129:3337-3342 (1991); Mau, S. E. et al., J Recept. Signal Transduct. Res., 15:311-323 (1995)). Ghrelin also activates MAP kinase and PI-3 kinase in HepG2 hepatoma cells (Murata, M. et al., J. Biol. Chem., 277:5667-5674 (2002)). To examine whether intracellular calcium release, phosphlipase C, PKC, MAP kinase and/or PI-3 kinase are involved in ghrelin-induced NF-κB activation, serum starved NCM-GHS-R cells are pretreated with BAPTA/AM (an intracellular calcium chelator), U73122 (a phospholipase C inhibitor), GF109203X (a broad PKC inhibitor), PD98059 (a MEK inhibitor), wortmannin or LY294002 (PI-3 kinase inhibitors) for 30 minutes and treated with ghrelin for an optimal time (as determined above). Nuclear extracts are prepared and DNA binding activity of NF-κB is then determined by EMSA. If GF109203X inhibits ghrelin-induced NF-κB activity, dominant negative mutants of different PKC isoforms are used to identify the specific PKC isoform involved in ghrelin-induced NF-κB. If wortmannin or LY294002 inhibits ghrelin-induced NF-κB activity, a dominant negative form of PI-3 kinase (p85-DN) is overexpressed to confirm these results. For this purpose, NCM-GHSR cells are infected with retroviruses expressing these mutant(s). Serum starved infected cells are then treated with ghrelin. DNA binding activity of NF-κB is determined by EMSA.

Anticipated Results

As discussed above, results herein show that ghrelin stimulates the NF-κB pathway in human colonic epithelial cells. Thus, it is reasonable to expect that the IκBα isoform will be degraded by GHS-R stimulation since IκBα is the major isoform of IκBα family (IκBα, IκBβ and IκBε).

Additionally, as discussed above, results herein also show that ghrelin-induced IL-8 promoter activity is inhibited by a pharmacological inhibitor CAPE and the endogenous inhibitor IκBα of the NF-κB pathway, indicating that the NF-κB pathway is involved in the pro-inflammatory effect. Molecular and biochemical approaches are used to examine whether ghrelin-induced IL-8 expression requires NF-κB, to examine whether ghrelin can activate this transcriptional factor and to identify the upstream molecules of NF-κB activated by ghrelin.

The teachings of all the articles, patents and patent applications cited herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

1. A method of inhibiting an inflammatory response in a tissue expressing ghrelin receptor positive cells, comprising administering to the tissue an agent that inhibits the signaling activity of the ghrelin receptor that mediates intestinal inflammation, thereby inhibiting the inflammatory response in the tissue.

2. (canceled)

3. The method of claim 1, wherein administering the agent comprises contacting the ghrelin receptor positive cells with the agent.

4. The method of claim 1, wherein the agent is selected from the group consisting of: a ghrelin antagonist, a ghrelin antibody or antigen-binding fragment thereof, a ghrelin derivative, a ghrelin inhibitor, a ghrelin receptor peptide or fragment, a ghrelin receptor inhibitor, a ghrelin receptor antagonist, a ghrelin receptor antibody or antigen-binding fragment thereof, a ghrelin analog, a ghrelin receptor peptide or fragment and a non-peptide ghrelin receptor antagonist.

5. The method of claim 1, wherein the agent is a competitive inhibitor of binding of ghrelin to the ghrelin receptor.

6. The method of claim 1, wherein the agent is a soluble isoform of the ghrelin receptor or a fragment thereof that retains the ability to bind to ghrelin.

7. The method of claim 1, wherein the agent inhibits binding to the ghrelin receptor, and wherein the agent is selected from the group consisting of: ghrelin antibodies, ghrelin receptor antagonists, ghrelin analogs and ghrelin derivatives.

8. The method of claim 7, wherein the ghrelin receptor antagonist is a peptide or peptide analog.

9. The method of claim 7, wherein the agent is an inhibitor of the ghrelin receptor binding activity of ghrelin.

10. The method of claim 1, where the inflammation is mediated by an autoimmune response, a parasite, a bacterium, a virus or a toxin.

11. The method of claim 10, where the toxin is produced by Clostridium difficile.

12. The method of claim 1, wherein the inflammation is of the small or large intestine.

13. The method of claim 1, wherein the intestinal inflammation is associated with:
a) an autoimmune response;
b) a parasitic infection;
c) inflammatory diarrhea;
d) inflammatory bowel disease;
e) Crohn's disease;
f) ulcerative colitis;
g) acute enterocolitis; or
h) chronic enterocolitis.

14-17. (canceled)

18. A method of treating inflammation associated with upregulation of ghrelin or ghrelin receptor in a mammal comprising administering to said mammal an effective amount of an agent that inhibits ghrelin activity, ghrelin binding to the ghrelin receptor or the signaling activity of the ghrelin receptor that mediates intestinal inflammation.

19. The method of claim 18, wherein the agent is selected from the group consisting of: a ghrelin antagonist, a ghrelin antibody or antigen-binding fragment thereof, a ghrelin derivative, a ghrelin inhibitor, a ghrelin receptor peptide or fragment, a ghrelin receptor inhibitor, a ghrelin receptor antagonist, a ghrelin receptor antibody or antigen-binding fragment thereof, a ghrelin analog, a ghrelin receptor peptide or fragment and a non-peptide ghrelin receptor antagonist.

20. A method for identifying a ghrelin antagonist or a ghrelin receptor antagonist comprising the steps of:
a) contacting cells expressing a ghrelin receptor with a candidate ghrelin antagonist or a candidate ghrelin receptor antagonist and with ghrelin;
b) determining MAP kinase phosphorylation in said cells which have been contacted with said candidate antagonist and with ghrelin;
c) comparing MAP kinase phosphorylation determined in step b) with MAP kinase phosphorylation in control cells which have been contacted with ghrelin and which have not been contacted with said candidate antagonist; and
d) selecting said candidate antagonist if MAP kinase phosphorylation determined in step b) is inhibited relative to MAP kinase phosphorylation in said control cells which have been contacted with ghrelin and which have not been contacted with said candidate antagonist,
wherein said candidate antagonist is identified as a ghrelin antagonist or a ghrelin receptor antagonist.

21. The method of claim 20 wherein steps a) to d) are repeated with a range of different concentrations of said candidate antagonist.

22. The method of claim 20 wherein in step a), said cells are contacted with said candidate antagonist prior to contact with ghrelin.

23. The method of claim 20 wherein said cells are intestinal epithelial cells.

24. The method of claim 20 wherein the cells expressing a ghrelin receptor is a cell line selected from the group consisting of: IEC-GHS-R and NCM460-GHSR.
